(19) United States
(12) Patent Application Publication
(10) Pub. No.: US 2013/0011480 A1
(43) Pub. Date: Jan. 10, 2013

(54) CYTOTOXIC THERAPY BY PROTON FLUX MODULATION

(76) Inventors: Mark A. ROSENBERG, Delray Beach, FL (US); James W. HILL, Mission Viejo, CA (US)

(21) Appl. No.: 13/464,896

(22) Filed: May 4, 2012

Related U.S. Application Data
(60) Provisional application No. 61/483,003, filed on May 5, 2011.

(51) Int. Cl. A61K 31/4439; A61K 31/7048; A61P 35/00; A61K 9/14; A61K 9/00; A61K 31/4965; A61K 31/433

(52) U.S. Cl. 424/489; 514/338; 514/255.06; 514/23; 514/363; 424/400

(57) ABSTRACT

Compositions and methods for treating cancer are described. Some of the methods include administering to a cancer patient in need thereof a substance, such as a carbonic anhydrase inhibitor, that at a therapeutic dose produces a metabolic acidosis in humans; and administering to the patient at least one of: (a) a monocarboxylate transport inhibitor; (b) a sodium-hydrogen exchange inhibitor; (c) a chloride-bicarbonate exchange inhibitor; or (d) a proton pump inhibitor; wherein the at least one of (a) through (d) is in an amount effective to induce selective cytotoxicity in cancer cells relative to noncancerous cells in humans.

### RELATED APPLICATIONS

This application claims the priority benefit of U.S. Provisional Application No. 61/483,003, filed on May 5, 2011, the entirety of which is hereby incorporated by reference.

### FIELD

Aspects of the subject technology involve cyotoxic therapy for diseases such as cancer.

### BACKGROUND

Cancer is a class of diseases in which a group of cells exhibits uncontrolled growth, invasion that destroys adjacent tissues, and sometimes metastasis, i.e., spread to other locations in the body via lymph or blood. These malignant properties of cancers differentiate them from benign tumors, which do not invade or metastasize.

Cancer causes of may be divided into two groups: environmental causes and hereditary genetic cause. Cancer is primarily an environmental disease, though genetics influence the risk of some cancers. Common environmental factors leading to cancer include tobacco, diet, obesity, viral infections, radiation, physical inactivity, and environmental pollutants. Environmental factors cause or enhance abnormalities in the genetic material of cells. Cell reproduction is a complex process normally regulated by several classes of genes, including oncogenes and tumor suppressor genes. Hereditary or acquired abnormalities in these regulatory genes can lead to the development of cancer. Approximately five to ten percent of cancers are apparently solely hereditary.

The presence of cancer can be suspected on the basis of symptoms or medical imaging findings. Definitive diagnosis of cancer requires microscopic examination of a tissue specimen. Many cancers can be treated with some combination of chemotherapy, radiotherapy, and surgery. The prognosis is influenced by the type of cancer and the extent of disease. Although a few types of cancer are more common in children than in adults, the overall risk of developing cancer increases with age. In 2007, cancer caused about 13% of human deaths worldwide (7.9 million). Cancer incidence and prevalence are rising as people live longer and lifestyles change in the developing world.

### SUMMARY

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause. The other clauses can be presented in a similar manner.

1. A pharmaceutical formulation, for treating cancer in a mammal, having active ingredients comprising at least two of
(a) a monocarboxylate transport inhibitor;
(b) a sodium-hydrogen exchange inhibitor;
(c) a chloride-bicarbonate exchange inhibitor;
(d) a carbonic anhydrase inhibitor; or
(e) a proton pump inhibitor;
wherein those of (a) through (e) that are in the formulation are in amounts effective in combination to induce selective cytotoxicity in cancer cells relative to noncancerous cells in members of the same species as the mammal.

2. The formulation of clause 1, wherein the mammal is human.

3. The formulation of clause 1, in a unit dose configured for enteral administration to the mammal at least every other day for at least three doses.

4. The formulation of clause 1, in a unit dose configured for enteral administration to the mammal at least every other day for at least one week.

5. The formulation of clause 1, in a unit dose configured for enteral administration to the mammal at least every other day for at least two weeks.

6. The formulation of clause 1, in a unit dose configured for enteral administration to the mammal at least every other day for at least three weeks.

7. The formulation of clause 1, in a unit dose configured for enteral administration to the mammal at least every other day for at least four weeks.

8. The formulation of clause 1, in a unit dose configured for enteral administration to the mammal at least every other day for at least three months.

9. The formulation of clause 1, further comprising at least one pharmaceutically acceptable excipient suitable for oral administration to a mammal.

10. The formulation of clause 1, wherein the proton pump inhibitor comprises a vacuolar-ATPase inhibitor.

11. The formulation of clause 1, wherein the proton pump inhibitor comprises a H+/K+-ATPase inhibitor.

12. The formulation of clause 1, wherein the proton pump inhibitor comprises at least one of omeprazole, lansoprazole, dexlansoprazole, esomeprazole, pantoprazole, rabeprazole, dorafem, or a bafilomycin.

13. The formulation of clause 1, wherein the monocarboxylate transport inhibitor comprises at least one of lonidamine, cinnamate, α-cyano-4-hydroxycinnamate (4-CIN), or a pharmacologically active derivative of 4-CIN.

14. The formulation of clause 1, wherein the sodium-hydrogen exchange inhibitor comprises at least one of amiloride, EIPA, or another pharmacologically active derivative of amiloride.

15. The formulation of clause 1, wherein the carbonic anhydrase inhibitor comprises at least one of methazolamide or acetazolamide.

16. The formulation of clause 1, wherein the chloride-bicarbonate exchange inhibitor comprises at least one of trifolcin, DIDS, diphenylamine-2-carboxylate, s3075, or levetiracetam.

17. The formulation of clause 1, wherein the chloride-bicarbonate exchange inhibitor comprises at least one of DIDS or a pharmacologically active derivative thereof.

18. The formulation of clause 1, in a form that is enterally administrable to the mammal.

19. The formulation of clause 1, in a form that is enterally administrable to the mammal and in a sufficient daily dose that, when administered daily, produces a metabolic acidosis in members of the same species as the mammal, the members having normal renal function.

20. The formulation of clause 19, comprising the carbonic anhydrase inhibitor and the proton pump inhibitor.

21. The formulation of clause 1, comprising the monocarboxylate transport inhibitor and the sodium-hydrogen exchange inhibitor.

22. The formulation of clause 1, comprising the monocarboxylate transport inhibitor and the chloride-bicarbonate exchange inhibitor.

23. The formulation of clause 1, comprising the monocarboxylate transport inhibitor and the carbonic anhydrase inhibitor.

24. The formulation of clause 1, comprising the monocarboxylate transport inhibitor and the proton pump inhibitor.

25. The formulation of clause 1, comprising the proton pump inhibitor and the sodium-hydrogen exchange inhibitor.

26. The formulation of clause 1, comprising the proton pump inhibitor and the chloride-bicarbonate exchange inhibitor.

27. The formulation of clause 1, comprising the proton pump inhibitor and the carbonic anhydrase inhibitor.

28. The formulation of clause 1, comprising the carbonic anhydrase inhibitor and the sodium-hydrogen exchange inhibitor.

29. The formulation of clause 1, comprising the carbonic anhydrase inhibitor and the chloride-bicarbonate exchange inhibitor.

30. The formulation of clause 1, comprising the sodium-hydrogen exchange inhibitor and the chloride-bicarbonate exchange inhibitor.

31. The formulation of clause 1, comprising at least three of (a) through (e).

32. The formulation of clause 1, comprising at least four of (a) through (e).

33. The formulation of clause 1, comprising each of (a) through (e).

34. The formulation of clause 1, comprising the monocarboxylate transport inhibitor, the sodium-hydrogen exchange inhibitor, and the chloride-bicarbonate exchange inhibitor.

35. The formulation of clause 1, comprising the monocarboxylate transport inhibitor, the chloride-bicarbonate exchange inhibitor, and the carbonic anhydrase inhibitor.

36. The formulation of clause 1, comprising the monocarboxylate transport inhibitor, the chloride-bicarbonate exchange inhibitor, and the proton pump inhibitor.

37. The formulation of clause 1, comprising the monocarboxylate transport inhibitor, the proton pump inhibitor, and the and the carbonic anhydrase inhibitor.

38. The formulation of clause 1, comprising the monocarboxylate transport inhibitor, the sodium-hydrogen exchange inhibitor, and the proton pump inhibitor.

39. The formulation of clause 1, comprising the monocarboxylate transport inhibitor, the sodium-hydrogen exchange inhibitor, and the carbonic anhydrase inhibitor.

40. The formulation of clause 1, comprising the monocarboxylate transport inhibitor, the sodium-hydrogen exchange inhibitor, and the carbonic anhydrase inhibitor.

41. The formulation of clause 1, comprising the monocarboxylate transport inhibitor, the sodium-hydrogen exchange inhibitor, and the carbonic anhydrase inhibitor.

42. The formulation of clause 1, comprising the monocarboxylate transport inhibitor, the sodium-hydrogen exchange inhibitor, and the carbonic anhydrase inhibitor.

43. The formulation of clause 1, comprising the monocarboxylate transport inhibitor, the chloride-bicarbonate exchange inhibitor, and the carbonic anhydrase inhibitor.

44. The formulation of clause 1, comprising the monocarboxylate transport inhibitor, the chloride-bicarbonate exchange inhibitor, and the proton pump inhibitor.

45. The formulation of clause 1, comprising the monocarboxylate transport inhibitor, the chloride-bicarbonate exchange inhibitor, and the carbonic anhydrase inhibitor.

46. The formulation of clause 1, comprising the monocarboxylate transport inhibitor, the chloride-bicarbonate exchange inhibitor, and the proton pump inhibitor.

47. The formulation of clause 1, comprising the monocarboxylate transport inhibitor, the proton pump inhibitor, and the carbonic anhydrase inhibitor.

48. The formulation of any of clause 1 through clause 47, further comprising a polymeric micelle that encases the active ingredients and releases them into extracellular fluid at pH below 7.3.

49. The formulation of clause 48, wherein the micelle releases the active ingredients into the extracellular fluid at pH below 7.2.

50. The formulation of clause 48, wherein the micelle releases the active ingredients into the extracellular fluid at pH below 7.1.

51. The formulation of clause 48, wherein the micelle releases the active ingredients into the extracellular fluid at pH below about 7.0.

52. The formulation of clause 48 through clause 51, wherein the micelle comprises at least one of a polyhistidine or a polysulfonamide.

53. The formulation of any of clause 48 through clause 52, wherein the micelle comprises at least one of a poly(ethylene glycol) or a poly(L-lactic acid).

54. The formulation of any of clause 1 through clause 53, further comprising an enteric coating that encases the active ingredients and resists degradation at a pH below about 5.0.

55. A method, of treating cancer in a patient, comprising:
(i) administering to a patient having cancer a substance that, at a therapeutic dose, produces a metabolic acidosis in humans; and
(ii) administering to the patient at least one of
(a) a monocarboxylate transport inhibitor;
(b) a sodium-hydrogen exchange inhibitor;
(c) a chloride-bicarbonate exchange inhibitor; or
(d) a proton pump inhibitor;
wherein the at least one of (a) through (d) is in an amount effective to induce selective cytotoxicity in cancer cells relative to noncancerous cells in humans.

56. The method of clause 55, wherein the proton pump inhibitor comprises a vacuolar ATPase inhibitor.

57. The method of clause 55, wherein the proton pump inhibitor comprises a H+/K+-ATPase inhibitor.

58. The method of clause 55, wherein the substance comprises a carbonic anhydrase inhibitor.

59. The method of clause 58, wherein the at least one of (a) through (d) comprises the proton pump inhibitor.

60. The method of clause 59, further comprising administering hyperthermia therapy to the patient.

61. The method of clause 60, wherein the hyperthermia therapy raises at least one of a core temperature or a rectal temperature of the patient to at least 103 degrees F.

62. The method of clause 59, further comprising, after steps (i) and (ii), administering at least one of chemotherapy, radiotherapy, or surgery to treat any residual cancer in the patient.

63. The method of clause 55, wherein the carbonic anhydrase inhibitor comprises methazolamide, administered in a dose greater than 200 mg in one day.

64. The method of clause 55, wherein the carbonic anhydrase inhibitor comprises methazolamide, administered in a dose greater than 200 mg per day.

65. The method of clause 55, wherein the carbonic anhydrase inhibitor comprises methazolamide, administered in a dose greater than 300 mg in one day.

66. The method of clause 55, wherein the carbonic anhydrase inhibitor comprises methazolamide, administered in a dose greater than 300 mg per day.

67. The method of clause 55, wherein the carbonic anhydrase inhibitor comprises acetazolamide, administered in a dose greater than 750 mg in one day.

68. The method of clause 55, wherein the carbonic anhydrase inhibitor comprises acetazolamide, administered in a dose greater than 750 mg per day.

69. The method of clause 55, wherein the carbonic anhydrase inhibitor comprises acetazolamide, administered in a dose greater than 1,000 mg in one day.

70. The method of clause 55, wherein the carbonic anhydrase inhibitor comprises acetazolamide, administered in a dose greater than 1,000 mg per day.

71. The method of clause 55, wherein the substance comprises a dinitrophenol.

72. The method of clause 55, comprising administering the proton pump inhibitor.

73. The method of clause 55, wherein the at least one of (a) through (d) comprises at least one of omeprazole, lansoprazole, dexlansoprazole, esomeprazole, pantoprazole, rabeprazole, dorafem, or a bafilomycin.

74. The method of clause 55, wherein the at least one of (a) through (d) comprises at least one of lonidamine, cinnamate, a-cyano-4-hydroxycinnamate (4-CIN), or a pharmacologically active derivative of 4-CIN.

75. The method of clause 55, wherein the at least one of (a) through (d) comprises at least one of amiloride, EIPA, or another pharmacologically active derivative of amiloride.

76. The method of clause 55, wherein the at least one of (a) through (d) comprises at least one of methazolamide or acetazolamide.

77. The method of clause 55, wherein the at least one of (a) through (d) comprises at least one of trifolcin, DIDS, diphenylamine-2-carboxylate, s3075, or levetiracetam.

78. The method of clause 55, wherein the at least one of (a) through (d) comprises at least one of DIDS or a pharmacologically active derivative thereof.

79. The method of clause 55, comprising administering the substance so as to produce a metabolic acidosis in the patient.

80. The method of clause 55, further comprising lowering the patient's serum pH.

81. The method of clause 80, further comprising administering an acid to the patient.

82. The method of clause 55, further comprising administering at least one of ammonium chloride or hydrochloric acid to the patient.

83. The method of clause 55, further comprising administering hyperthermia therapy to the patient.

84. The method of clause 83, wherein the hyperthermia therapy raises at least one of a core temperature or a rectal temperature of the patient to at least 103 degrees F.

85. The method of clause 55, wherein the substance is administered enterally to the patient.

86. The method of clause 55, wherein the at least one of (a) through (d) are administered enterally to the patient.

87. The method of clause 55, wherein the substance and the at least one of (a) through (d) are each administered enterally to the patient.

88. The method of clause 55, wherein the substance is administered enterally at a daily dose that, when continued daily, produces the metabolic acidosis.

89. The method of clause 55, wherein the at least one of (a) through (d) comprises the monocarboxylate transport inhibitor or the sodium-hydrogen exchange inhibitor.

90. The method of clause 55, wherein the at least one of (a) through (d) comprises the monocarboxylate transport inhibitor or the chloride-bicarbonate exchange inhibitor.

91. The method of clause 55, wherein the at least one of (a) through (d) comprises the monocarboxylate transport inhibitor or the proton pump inhibitor.

92. The method of clause 55, wherein the at least one of (a) through (d) comprises the proton pump inhibitor or the sodium-hydrogen exchange inhibitor.

93. The method of clause 55, wherein the at least one of (a) through (d) comprises the proton pump inhibitor or the chloride-bicarbonate exchange inhibitor.

94. The method of clause 55, wherein the at least one of (a) through (d) comprises the sodium-hydrogen exchange inhibitor or the chloride-bicarbonate exchange inhibitor.

95. The method of clause 55, comprising administering to the patient at least three of (a) through (d).

96. The method of clause 55, comprising administering to the patient at least four of (a) through (d).

97. The method of clause 55, comprising administering to the patient each of (a) through (d).

98. The method of clause 55, wherein the at least one of (a) through (d) comprises the monocarboxylate transport inhibitor, the sodium-hydrogen exchange inhibitor, or the chloride-bicarbonate exchange inhibitor.

99. The method of clause 55, wherein the at least one of (a) through (d) comprises the monocarboxylate transport inhibitor, the chloride-bicarbonate exchange inhibitor, or the proton pump inhibitor.

100. The method of clause 55, wherein the at least one of (a) through (d) comprises the monocarboxylate transport inhibitor, the sodium-hydrogen exchange inhibitor, or the proton pump inhibitor.

101. The method of clause 55, wherein the at least one of (a) through (d) comprises the monocarboxylate transport inhibitor, the chloride-bicarbonate exchange inhibitor, or the proton pump inhibitor.

102. The method of clause 55, wherein the at least one of (a) through (d) comprises the monocarboxylate transport inhibitor, the chloride-bicarbonate exchange inhibitor, or the proton pump inhibitor.

103. The method of clause 55, further comprising administering a carbohydrate to the patient.

104. The method of clause 55, further comprising administering intravenous glucose to the patient.

105. The method of clause 55, further comprising administering glucose and insulin to the patient.

106. The method of clause 55, further comprising administering lactate to the patient.

107. The method of clause 55, further comprising administering a dinitrophenol to the patient.

108. A pharmaceutical composition, enterally deliverable to an animal, comprising a bioactive agent at least partially surrounded by three layers;
wherein the first layer is the outermost of the layers and comprises a first material that is (a) substantially insoluble in aqueous media below a pH of about 5.0, and (b) substantially soluble in aqueous media above a pH of about 6.0;
wherein the second layer lies between the first and the third layer and comprises a second material that erodes at a predetermined rate in aqueous media between a pH of about 7.2 and about 7.6;
wherein the third layer is the innermost of the three layers and is configured (a) not to erode above a pH of about 7.4, and (b) to erode below a pH of about 7.3, thereby releasing the bioactive agent to a target tissue of the animal from within the third layer.

109. The composition of clause 108, wherein the third layer comprises a third material that is (a) substantially insoluble in aqueous media above a pH of about 7.4, and (b) substantially soluble in aqueous media below a pH of about 7.3.

110. The composition of clause 108, wherein the first material is (a) substantially insoluble in aqueous media below a pH of about 5.0, and (b) substantially soluble in aqueous media above a pH of about 6.5.

111. The composition of clause 108, wherein the second and third layers are configured such that the third material releases the bioactive agent to the target tissue after the third layer has been absorbed across a gut wall of the animal.

112. The composition of clause 108, wherein the second layer is configured such that the second material erodes within the animal's body after the second layer is absorbed across a gut wall of the animal.

113. The composition of clause 108, wherein at least one of the second and third layers is absorbable across a gut wall of the animal.

114. The composition of clause 108, further comprising a pharmaceutically acceptable carrier of the bioactive agent.

115. The composition of clause 108, further comprising a coupling agent located in or on at least one of the second and third layers, wherein the coupling agent is configured to couple to at least part of a molecule at the target tissue.

116. The composition of clause 115, wherein the coupling agent comprises an antibody.

117. The composition of clause 115, wherein the coupling agent comprises a protein.

118. The composition of clause 108, further comprising a pharmaceutically acceptable carrier of the bioactive agent.

119. A pharmaceutical composition orally deliverable to a human subject, comprising a bioactive agent at least partially surrounded by three layers;
wherein the first layer is the outermost of the three layers and comprises a first material that resists degradation by human stomach acid and enzymes;
wherein the second layer lies between the first and the third layer and comprises a second material that degrades at a predetermined rate;
wherein the third layer is the innermost of the three layers and comprises a third material that substantially releases the bioactive agent into extracellular fluid of the subject when a pH of the extracellular fluid is below about 7.4.

120. The composition of clause 119, wherein at least one of the second and third layers is absorbable across an intestinal wall of the subject.

121. The composition of clause 119, wherein the third material substantially releases the bioactive agent when the pH is below 7.3.

122. The composition of clause 119, wherein the third material substantially releases the bioactive agent when the pH is below 7.2.

123. The composition of clause 119, wherein the third material substantially releases the bioactive agent when the pH is below 7.1.

124. The composition of clause 119, wherein the third material substantially releases the bioactive agent when the pH is below 7.0.

125. The composition of clause 119, wherein the third material is (a) substantially water-insoluble above a pH of about 7.4, and (b) substantially water-soluble below a pH of about 7.3.

126. The composition of clause 119, wherein the first material is (a) substantially insoluble in aqueous media below a pH of about 5.0, and (b) substantially soluble in aqueous media above a pH of about 6.5.

127. The composition of clause 119, wherein the second and third layers are configured such that the third material releases the bioactive agent to the target tissue after the third layer has been absorbed across a gut wall of the animal.

128. The composition of clause 119, wherein the second layer is configured such that the second material erodes within the animal's body after the second layer is absorbed across a gut wall of the animal.

129. The composition of clause 119, wherein at least one of the second and third layers is absorbable across a gut wall of the animal.

130. The composition of clause 119, further comprising a pharmaceutically acceptable carrier of the bioactive agent.

131. The composition of clause 119, further comprising a coupling agent located in or on at least one of the second and third layers, wherein the coupling agent is configured to couple to at least part of a molecule at the target tissue.

132. The composition of clause 119, wherein the coupling agent comprises an antibody.

133. The composition of clause 119, wherein the coupling agent comprises a protein.

134. The composition of clause 119, further comprising a pharmaceutically acceptable carrier of the bioactive agent.

135. A pharmaceutical composition enterally deliverable to an animal, comprising a bioactive agent at least partially surrounded by three layers;
wherein the first layer is the outermost of the three layers and comprises a first material that is (a) substantially insoluble in aqueous media below a pH of about 5.0, and (b) substantially soluble in aqueous media above a pH of about 6.0;
wherein the second layer lies between the first and the third layer and comprises a second material that erodes at a predetermined rate in aqueous media between a pH of about 7.2 and about 7.6;
wherein the third layer is the innermost of the three layers and comprises a third material that is (a) substantially soluble in aqueous media above a pH of about 7.4, and (b) substantially insoluble in aqueous media below a pH of about 7.3, permitting release of the bioactive agent to a target tissue of the animal from within the third layer.

136. The composition of clause 135, wherein the first material is (a) substantially insoluble in aqueous media below a pH of about 5.0, and (b) substantially soluble in aqueous media above a pH of about 6.5.

137. The composition of clause 135, wherein the third material is (a) substantially soluble in aqueous media above a pH of about 7.5, and (b) substantially insoluble in aqueous media below a pH of about 7.2, permitting release of the bioactive agent to a target tissue of the animal from within the third layer.

138. A pharmaceutical formulation, for treating cancer in a mammal, having active ingredients comprising:
(a) a carbonic anhydrase inhibitor;
(b) a proton pump inhibitor; and
(c) at least one of a copper chelator, a platinum-based chemotherapeutic, an autophagy modulator, a glutaminolysis inhibitor, a glycolysis inhibitor, or a somatostatin receptor binding agent;
wherein those of (a) through (c) that are in the formulation are in amounts effective in combination to induce selective cytotoxicity in cancer cells relative to noncancerous cells in members of the same species as the mammal.

139. The formulation of clause 138, wherein the mammal is human.

140. The formulation of clause 138, in a unit dose configured for enteral administration to the mammal at least every other day for at least three doses.

141. The formulation of clause 138, in a unit dose configured for enteral administration to the mammal at least every other day for at least one week.

142. The formulation of clause 138, in a unit dose configured for enteral administration to the mammal at least every other day for at least two weeks.

143. The formulation of clause 138, in a unit dose configured for enteral administration to the mammal at least every other day for at least three weeks.

144. The formulation of clause 138, in a unit dose configured for enteral administration to the mammal at least every other day for at least four weeks.

145. The formulation of clause 138, in a unit dose configured for enteral administration to the mammal at least every other day for at least three months.

146. The formulation of clause 138, further comprising at least one pharmaceutically acceptable excipient suitable for oral administration to a mammal.

147. The formulation of clause 138, wherein the proton pump inhibitor comprises a vacuolar-ATPase inhibitor.

148. The formulation of clause 138, wherein the proton pump inhibitor comprises a H+/K+-ATPase inhibitor.

149. The formulation of clause 138, wherein the proton pump inhibitor comprises at least one of omeprazole, lansoprazole, dexlansoprazole, esomeprazole, pantoprazole, rabeprazole, dorafem, or a bafilomycin.

150. The formulation of clause 138, wherein the carbonic anhydrase inhibitor comprises at least one of methazolamide or acetazolamide.

151. The formulation of clause 138, in a form that is enterally administrable to the mammal.

152. The formulation of clause 138, in a form that is enterally administrable to the mammal and in a sufficient daily dose that, when administered daily, produces a metabolic acidosis in members of the same species as the mammal, the members having normal renal function.

153. The formulation of clause 138, comprising the carbonic anhydrase inhibitor, the proton pump inhibitor and the copper chelator.

154. The formulation of clause 138, wherein the copper chelator is selected from the group consisting of penicillamine (Cuprimine®, Depen®), trientine hydrochloride (also known as triethylenetetramine hydrochloride, or Syprine®), dimercaprol, diethyldithiocarbamate (e.g., sodium diethyldithiocarbamate), bathocuproine sulfonate, and tetrathiomolybdate (e.g., ammonium tetrathiomolybdate).

155. The formulation of clause 138, comprising the carbonic anhydrase inhibitor, the proton pump inhibitor and the platinum-based chemotherapeutic.

156. The formulation of clause 138, wherein the platinum-based chemotherapeutic is selected from the group consisting of cisplatin, carboplatin, oxaliplatin, iproplatin, tetraplatin, lobaplatin, dicycloplatin (DCP), PLD-147, JM118, JM216, JM335, and satraplatin.

157. The formulation of clause 138, comprising the carbonic anhydrase inhibitor, the proton pump inhibitor and the autophagy modulator.

158. The formulation of clause 138, wherein the autophagy modulator is selected from the group consisting of chloroquine, hydroxychloroquine (Plaquenil®), 3-methyladenine and rapamyc in.

159. The formulation of clause 138, comprising the carbonic anhydrase inhibitor, the proton pump inhibitor and the glutaminolysis inhibitor.

160. The formulation of clause 138, wherein the glutaminolysis inhibitor is selected from the group consisting of amino-oxyacetate (AOA), sodium phenylbutyrate, phenylbutyrate, phenylacetate, and 3,7-bis(dimethylamino)-phenazathionium chloride (methylene blue).

161. The formulation of clause 138, comprising the carbonic anhydrase inhibitor, the proton pump inhibitor and the glycolysis inhibitor.

162. The formulation of clause 138, wherein the glycolysis inhibitor is selected from the group consisting of 2-deoxy-D-glucose (2DG), oxamate and analogs thereof.

163. The formulation of clause 138, comprising the carbonic anhydrase inhibitor, the proton pump inhibitor and somatostatin receptor binding agent.

164. The formulation of clause 138, wherein the somatostatin receptor binding agent is selected from the group consisting of octreotide, vapreotide and seglitide.

165. The formulation of any of clause 138 through clause 164, further comprising a polymeric micelle that encases the active ingredients and releases them into extracellular fluid at pH below 7.3.

166. The formulation of clause 165, wherein the micelle releases the active ingredients into the extracellular fluid at pH below 7.2.

167. The formulation of clause 165, wherein the micelle releases the active ingredients into the extracellular fluid at pH below 7.1.

168. The formulation of clause 165, wherein the micelle releases the active ingredients into the extracellular fluid at pH below about 7.0.

169. The formulation of clause 165 through clause 168, wherein the micelle comprises at least one of a polyhistidine or a polysulfonamide.

170. The formulation of any of clause 165 through clause 169, wherein the micelle comprises at least one of a poly(ethylene glycol) or a poly(L-lactic acid).

171. The formulation of any of clause 135 through clause 170, further comprising an enteric coating that encases the active ingredients and resists degradation at a pH below about 5.0.

172. A method, of treating cancer in a patient, comprising:
(i) administering to a patient having cancer a substance that, at a therapeutic dose, produces a metabolic acidosis in humans; and
(ii) administering to the patient (a) a proton pump inhibitor and (b) at least one of a copper chelator, a platinum-based chemotherapeutic, an autophagy modulator, a glutaminolysis inhibitor, a glycolysis inhibitor, or a somatostatin receptor binding agent;
wherein the (a) and (b) are in an amount effective to induce selective cytotoxicity in cancer cells relative to noncancerous cells in humans.

173. The method of clause 172, wherein the proton pump inhibitor comprises a vacuolar ATPase inhibitor.

174. The method of clause 172, wherein the proton pump inhibitor comprises a H+/K+-ATPase inhibitor.

175. The method of clause 172, wherein the substance comprises a carbonic anhydrase inhibitor.

176. The method of clause 172, further comprising administering hyperthermia therapy to the patient.

177. The method of clause 172, wherein the hyperthermia therapy raises at least one of a core temperature or a rectal temperature of the patient to at least 103 degrees F.

178. The method of clause 172, further comprising, after steps (i) and (ii), administering at least one of chemotherapy, radiotherapy, or surgery to treat any residual cancer in the patient.

179. The method of clause 172, further comprising administering a ketogenic diet before, after or concurrently with the steps (i) and (ii).

180. The method of clause 175, wherein the carbonic anhydrase inhibitor comprises methazolamide, administered in a dose greater than 200 mg in one day.

181. The method of clause 175, wherein the carbonic anhydrase inhibitor comprises methazolamide, administered in a dose greater than 200 mg per day.

182. The method of clause 175, wherein the carbonic anhydrase inhibitor comprises methazolamide, administered in a dose greater than 300 mg in one day.

183. The method of clause 175, wherein the carbonic anhydrase inhibitor comprises methazolamide, administered in a dose greater than 300 mg per day.

184. The method of clause 175, wherein the carbonic anhydrase inhibitor comprises acetazolamide, administered in a dose greater than 750 mg in one day.

185. The method of clause 175, wherein the carbonic anhydrase inhibitor comprises acetazolamide, administered in a dose greater than 750 mg per day.

186. The method of clause 175, wherein the carbonic anhydrase inhibitor comprises acetazolamide, administered in a dose greater than 1,000 mg in one day.

187. The method of clause 175, wherein the carbonic anhydrase inhibitor comprises acetazolamide, administered in a dose greater than 1,000 mg per day.

188. The method of clause 172, wherein the substance comprises a dinitrophenol.

189. The method of clause 172, further comprising lowering the patient's serum pH.

190. The method of clause 172, further comprising administering an acid to the patient.

191. The method of clause 172, further comprising administering at least one of ammonium chloride or hydrochloric acid to the patient.

192. The method of clause 172, further comprising administering hyperthermia therapy to the patient.

193. The method of clause 172, wherein the hyperthermia therapy raises at least one of a core temperature or a rectal temperature of the patient to at least 103 degrees F.

194. The method of clause 172, wherein the substance is administered enterally to the patient.

195. The method of clause 172, wherein the at least one of (a) or (b) are administered enterally to the patient.

196. The method of clause 172, wherein the substance and the at least one of (a) or (b) are each administered enterally to the patient.

197. The method of clause 172, wherein the substance is administered enterally at a daily dose that, when continued daily, produces the metabolic acidosis.

198. The method of clause 172, further comprising administering a carbohydrate to the patient.

199. The method of clause 172, further comprising administering intravenous glucose to the patient.

200. The method of clause 172, further comprising administering glucose and insulin to the patient.

201. The method of clause 172, further comprising administering lactate to the patient.

202. The method of clause 172, further comprising administering a dinitrophenol to the patient.

203. The method of clause 172, further comprising administering a ketogenic diet to the patient.

204. The method of clause 172, wherein the copper chelator is selected from the group consisting of penicillamine (Cuprimine®, Depen®), trientine hydrochloride (also known as triethylenetetramine hydrochloride, or Syprine®), dimercaprol, diethyldithiocarbamate (e.g., sodium diethyldithiocarbamate), bathocuproine sulfonate, and tetrathiomolybdate (e.g., ammonium tetrathiomolybdate).

205. The method of clause 172, wherein the platinum-based chemotherapeutic is selected from the group consisting of cisplatin, carboplatin, oxaliplatin, iproplatin, tetraplatin, lobaplatin, dicycloplatin (DCP), PLD-147, JMI 18, JM216, JM335, and satraplatin.

206. The method of clause 172, wherein the autophagy modulator is selected from the group consisting of chloroquine, hydroxychloroquine (Plaquenil®), 3-methyladenine and rapamyc in.

207. The method of clause 172, wherein the glutaminolysis inhibitor is selected from the group consisting of amino-oxyacetate (AOA), sodium phenylbutyrate, phenylbutyrate, phenylacetate, and 3,7-bis(dimethylamino)-phenazathionium chloride (methylene blue).

208. The method of clause 172, wherein the glycolysis inhibitor is selected from the group consisting of 2-deoxy-D-glucose (2DG), oxamate and analogs thereof.

209. The method of clause 172, wherein the somatostatin receptor binding agent is selected from the group consisting of octreotide, vapreotide and seglitide.

### BRIEF DESCRIPTION OF THE DRAWINGS

FIG. 1 shows Resolving the Autophagy Paradox in Cancer Therapy: the large black arrow signifies energy transfer (E.T.) from stromal cancer associated fibroblasts (CAFs) to epithelial cancer cells, via stromal autophagy/mitophagy. Thus, inhibition of autophagy in the tumor stroma would be expected to halt or reverse tumor growth. This could explain the effectiveness of known autophagy inhibitors as anti-tumor agents, such as chloroquine, hydroxychloroquine (Plaquenil®) and 3-methyladenine (Upper panel). Conversely, induction of autophagy in epithelial cancer cells would also be expected to block or inhibit tumor growth (Lower panel); E.T., energy transfer; AM+, increased autophagy/mitophagy; AM-, decreased autophagy/mitophagy; Rx, therapy with autophagy promoters or inhibitors.

### DETAILED DESCRIPTION

In the following detailed description, numerous specific details are set forth to provide a full understanding of the subject technology. It will be apparent, however, to one ordinarily skilled in the art that the subject technology may be practiced without some of these specific details. In other instances, well-known structures and techniques have not been shown in detail so as not to obscure the subject technology.

A phrase such as “an aspect” does not imply that such aspect is essential to the subject technology or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa. A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure. A phrase such “an embodiment” may refer to one or more embodiments and vice versa. A phrase such as “a configuration” does not imply that such configuration is essential to the subject technology or that such configuration applies to all configurations of the subject technology. A disclosure relating to a configuration may apply to all configurations, or one or more configurations. A configuration may provide one or more examples of the disclosure. A phrase such as “a configuration” may refer to one or more configurations and vice versa.

Cancer and Acid-Base Considerations

Manipulation of the extracellular and/or intracellular pH of tumors may have considerable potential in cancer therapy. The extracellular space of most tumors is mildly acidic, owing principally to large production of lactic acid. Aerobic glycolysis, attributable largely to chronic activation of hypoxia-inducible factor-1 (HIF-1), as well as tumor hypoxia, are chiefly responsible for this phenomenon. Tumor acidity tends to correlate with cancer aggressiveness; in part, this reflects the ability of HIF-1 to promote invasiveness and angiogenesis. But extracellular acidity per se apparently boosts the invasiveness and metastatic capacity of cancer cells. Moreover, this acidity renders cancer cells relatively resistant to the high proportion of chemotherapeutic drugs that are mildly basic, and may impede immune rejection of tumors.

Strategies for raising the extracellular pH of tumors has therapeutic utility in some cases. In rodents, oral administration of sodium bicarbonate can raise the extracellular pH of tumors, an effect associated with inhibition of metastasis and improved responsiveness to certain cytotoxic agents; clinical application of this strategy is feasible. Drugs that inhibit proton pumps in cancer cells may alleviate extracellular tumor acidity while lowering the intracellular pH of cancer cells; reduction of intracellular pH slows proliferation and promotes apoptosis in various cancer cell lines. Well-tolerated doses of the proton pump inhibitor esomeprazole markedly impede tumor growth and prolong survival in nude mice implanted with a human melanoma.

One can exploit the glycolysis of cancers in hyperacidification therapies; intense intracellular acidification of cancer cells achieved by induced hyperglycemia, concurrent administration of proton pump inhibitor drugs, and possibly dinitrophenol, can kill cancer cells directly or potentiate their responsiveness to adjunctive measures. A similar strategy, with or without proton pump inhibition, could be employed to maximize extracellular tumor acidity. This also assists in enabling tumor-selective release of cytotoxic drugs encased in pH-sensitive nanoparticles.

Four categorical strategies can be implemented: (1) alkalizer therapy that increases the pH of the extracellular space; (2) proton pump inhibition that decreases the intracellular pH, while increasing the extracellular pH; (3) acute intracellular acidification that kills cancer cells directly or potentiates their sensitivity to adjuvant measures; and (4) acute extracellular acidification that enables tumor-selective release of cytotoxic drugs encased in pH-sensitive nanoparticles.

A fifth strategy can also be implemented: extracellular acidification, such as by inducing, or attempting to induce, a metabolic acidosis using oral drugs taken over days to months, combined with intracellular acidification, e.g., with proton pump inhibitors, that kills cancer cells directly or potentiates their sensitivity to adjuvant measures.

At least six molecular targets can be employed for inhibition of proton extrusion from cancer cells, producing metabolically directed anticancer treatment. Examples of inhibitor drugs are provided, targeting different proton or bicarbonate transport mechanisms at the sites of their activity.

Abbreviations in this disclosure include: NHE1: Na+/H+ exchanger: HMA: 5-(N,N-hexamethylene)-amiloride, DMA: 5-(N,N-dimethyl)amiloride; HIF-1: hypoxia-inducible factor; MCT1: monocarboxylate transporter or H+-lactate co-transporter; CAIX: carbonic anhydrase IX; V−H+-ATPase: vacuolar H+-ATPase; VEGF: vasoendothelial growth factor; UKt-PA: urokinase-type plasminogen activator; P-gp: P-glycoprotein; MDR: multiple drug resistance; pHi: intracellular pH; pHe: extracellular/interstitial tumoral pH.

Extracellular Acidity: Indicator and Mediator of Cancer Aggressiveness

Most cancers are characterized by wasteful glycolytic conversion of glucose to lactic acid, even when sufficient oxygen is available to support efficient mitochondrial respiration. The extent to which this phenomenon is expressed tends to correlate with tumor aggressiveness. The aerobic glycolysis of cancer cells is commonly attributable to chronic overactivation of the transcription factor hypoxia-inducible factor-1 (HIF-1), which boosts expression of a range of glycolytic enzymes and pyruvate dehydrogenase kinase-1 (which functions to inhibit pyruvate dehydrogenase and thus expedite conversion of pyruvate to lactate) and promotes mitochondrial autophagy.

Tumor production of lactic acid is also driven by anaerobic glycolysis in tumor regions that are hypoxic. Owing to avid production of lactic acid, the extracellular space of most tumors is mildly acidic, with the greatest degree of acidity encountered in the tumor core. Cancer cells, however, usually maintain a normal intracellular pH, owing to proton pumps and intracellular buffers.

The degree to which pH is depressed in tumors, as mirrored by their lactate levels, tends to correlate with prognosis, the more acidic tumors being associated with poorer outcome. In part, this phenomenon may reflect the fact that tumor acidity is serving as a marker for HIF-1 activation, which works in a variety of complementary ways to boost tumor capacity for invasion, metastasis, angiogenesis, and chemoresistance. However, there is increasing evidence that extracellular acidity per se contributes to the aggressiveness of cancer cells, boosting extracellular proteolytic activities, expression of pro-angiogenic factors, and metastatic capacity. Homeostatically, this makes good sense—extracellular acidity, like hypoxia, is a typical consequence of suboptimal perfusion, and it is not surprising that cells have evolved to sense this acidity and take appropriate countermeasures.

Cultivation of various types of cancer cells under the mildly acidic conditions that prevail in many tumors has been reported to boost transcription of the angiogenic factors vascular endothelial growth factor (VEGF) and interleukin (IL)-8, increase extracellular release and/or expression of key proteases such as cathepsin B, matrix metalloproteinases-2 and -9, and to amplify the invasiveness and metastatic capacity of cancer cells, in vitro and in vivo. In one particularly striking study, incubation of various human melanoma cell lines at pH 6.8 (compared with 7.4) for 48 hours approximately doubled the yield of lung metastases following their intravenous administration in nude mice. An analogous impact of prolonged exposure to extracellular acidity on the invasiveness and migratory activity of human melanoma cells in vitro has also been reported. The effect of extracellular acidity on HIF-1 activity appears so far to have received little study.

Activated transcription of VEGF and IL-8 under acid conditions has been traced to increased activity of nuclear factor-kappaB and activator protein-1 in certain cancer cell lines. Increased extracellular proteolytic activity appears to reflect, in part, an increased tendency of lysosomes to migrate to the cell periphery and discharge their contents via exocytosis. Acidification of the extracellular space in tumors can also contribute to chemoresistance. Because many cytotoxic cancer drugs are mildly basic, their increased protonation in the extracellular space of tumors would be expected to impede their transit through cell membranes, rendering cancer cells less susceptible to their effects. Moreover, extracellular lactic acid can suppress the tumoricidal activity of cytotoxic T lymphocytes and natural killer cells; it also inhibits lymphocyte proliferation and dendritic cell maturation. These immunosuppressive effects appear not to be mediated by acidity per se, but by influx of lactic acid via a lactate/H+ co-transporter that under neutral conditions functions to remove lactic acid from leukocytes.

Because tumor acidity appears to make a meaningful contribution to cancer aggressiveness, chemoresistance, and evasion of immune rejection, measures for normalizing the pH of tumors may have therapeutic utility. Aerobic glycolysis and tumor acidification could be suppressed by measures that inhibit the activity of HIF-1. Various practical strategies for achieving this may be currently available, and new drugs are being developed that target this transcription factor. However, alternative measures for ameliorating the extracellular acidity of tumors have been proposed. Novel strategies for exploiting the aerobic glycolysis of tumors in cancer therapy may also prove feasible.

Systemic Buffering: Increasing Extracellular pH

Gillies et al. have shown that dietary measures that boost the bicarbonate level of plasma can elevate the subnormal pH of tumors to some degree, without notably influencing the pH of blood or healthy tissues. The failure of oral bicarbonate to influence the pH of plasma presumably reflects the fact that a physiological buffer tends to drive pH to the physiological value of 7.4.

This strategy has clinical potential. In nude mice implanted with a human breast cancer, chronic oral administration of sodium bicarbonate, while not influencing the expansion of primary tumors, markedly reduces the number and size of metastases in lung, visceral organs, and lymph nodes. These findings thus raise the possibility that systemic buffering, achieved by oral administration of high doses of agents such as sodium bicarbonate or trisodium citrate—or possibly even a natural diet of low-to-moderate protein content, but high in potassium-rich fruits, vegetables, and juices—could dampen the aggressiveness of certain cancers by partially alleviating their extracellular acidity. Whether this strategy would influence transcriptional activity of HIF-1 is unclear, but it evidently would tend to counteract one of the key pathogenic downstream consequences of HIF-1 overactivation. It is curious to note that alkalizing diets have long been recommended by naturopathic physicians as a strategy for slowing cancer spread, and that oral administration of sodium bicarbonate—albeit in doses that likely are clinically suboptimal—has also recently gained popularity as an alternative cancer therapy.

In a sodium bicarbonate breast cancer study, mice received about 0.84 mEq Na daily; assuming that the mice weighed about 20 g, and normalizing by the ¾th power of relative weights, the equivalent dose in a 70 kg human would be 378 mEq, which corresponds to a daily dose of 31.75 g sodium bicarbonate or 32.5 g trisodium citrate. Some have implemented an alkalinizing therapy with cancer patients. Large acute doses of either sodium bicarbonate or trisodium citrate (which have been studied as aids to exercise performance, usually at 40-60 mg/kg) can induce temporary nausea and diarrhea; it therefore is prudent to administer the daily dose gradually throughout the day. Moreover, gradual administration should achieve a more even elevation of tumor extracellular pH than could be achieved with bolus doses. Possible protocols include: add 500 mL (about 2 cups) of water to a small teapot and stir in two rounded teaspoons (about 12 g) of sodium bicarbonate or trisodium citrate plus one packet of sweetener.

Active patients can prepare this beverage in a 500-mL water bottle. This is to be consumed gradually over an hour or more. If this procedure is followed three times daily (e.g., in mid-morning, mid-afternoon, and late evening), about 36 g of sodium bicarbonate or trisodium citrate will be provided daily. If desired, this fluid can be heated to make tea or herb teas, or can be flavored with a Crystal Light™-type product.

Sodium bicarbonate has the advantage that it is quite inexpensive and readily available; trisodium citrate may be less likely to promote intestinal gas.

Exploiting Proton Pump Inhibitors: Decreasing Intracellular pH

As an alternative or adjunctive strategy for correcting the extracellular acidity of tumors, some have explored inhibition of the membrane ion pumps that maintain an alkaline intracellular pH by extruding protons or importing bicarbonate ions. Many cancers express extracellular forms of carbonic anhydrase—CAIX and CAII—which acidify the extracellular space while generating bicarbonate that can be imported. A Na+/H+ exchanger (NHE), of which several isoforms exist, is a prominent mediator of proton extrusion; the NHE1 isoform is ubiquitously expressed. Proton extrusion is also achieved by the vacuolar H+-ATPase (V-ATPase), which hydrolyzes ATP to drive proton pumping. Although the chief physiological role of this pump is to acidify intracellular vacuoles such as lysosomes and endosomes, it is also expressed in the plasma membrane of many cancer cells, particularly those with metastatic capacity. Moreover, the protons pumped into vacuoles often reach the extracellular space when these vacuoles fuse with the plasma membrane and extrude their contents.

A Na+-dependent Cl−/HCO3− exchanger also functions to maintain an alkaline intracellular pH. Proton pump inhibition tends to decrease intracellular pH, as it raises that of the extracellular space. The ameliorative impact on extracellular acidity tends to be sustained (despite the fact that the rate of lactate generation must ultimately match the rate of lactate export), presumably because intracellular acidity tends to suppress the efficiency of glycolysis. 52 The intracellular acidification associated with proton pump inhibition can have an impact on cancer cell behavior, independent of that of the associated elevation of extracellular pH. Indeed, proton pump inhibitors exert anti-proliferative and pro-apoptotic effects on certain cancer cell lines; furthermore, intracellular acidification has been shown to enhance the killing efficacy of hyperthermia (42° C.+) as well as the apoptotic response to tumor necrosis factor related apoptosis-inducing ligand (TRAIL).

Although different agents have been employed as proton pump inhibitors in cell culture or rodent studies, few have been used clinically. Notable exceptions are the proton pump inhibitor drugs (PPIs; e.g., omeprazole, esomeprazole) used clinically to suppress gastric acidity.

When activated by mildly acidic conditions, these drugs can inhibit the V-ATPase by a covalent interaction. As emphasized by De Milito et al, the particular merit of these drugs is that their impact can be tumor-specific, as they are activated in the mildly acidic extracellular space of tumors. Other agents, such as bafilomycin, that act to inhibit V-ATPase, are tissue non-selective and have been found to have systemic toxicity. In vitro, V-ATPase inhibitors have exerted anti-proliferative, pro-apoptotic, and thermosensitizing effects. In vivo, non-toxic doses of PPIs, analogous to those used clinically in Zollinger-Ellison syndrome, can suppress the growth of a human melanoma in nude mice, an effect associated with a near-doubling of survival time. Knockdown of V-ATPase expression achieved via small interfering RNA in a human hepatocellular carcinoma can markedly slow the growth and impede the metastatic spread of this cancer in nude mice.

Preadministration of omeprazole notably can potentiate the growth-retardant impact of cisplatin on a human melanoma in nude mice, presumably at least in part because cisplatin is a mildly basic drug whose intracellular uptake is impaired by the acidic extracellular milieu of tumors.

Another key mediator of proton extrusion is sodium-hydrogen ion exchange inhibitor 1, or NHE1. This pump can be inhibited by supraclinical concentrations of the diuretic amiloride. A derivative of amiloride, EIPA, is about 200 times more potent in this regard, but has not been developed for clinical use. One can employ the NHE1 inhibitor cariporide, which has been taken to phase III trials as a drug for decreasing risk of myocardial infarction (MI) subsequent to coronary bypass surgery. This agent is unlikely may never be approved for this purpose, as it was found to modestly increase cerebrovascular mortality while decreasing MI risk. Nonetheless, a well-tolerated dosage schedule has been established, so this drug could be developed as a cancer therapeutic if its utility could be established in rodent studies. A number of studies have established that this agent, often used in conjunction with an inhibitor of the Cl−/HCO3-exchanger, can lower the intracellular pH of cancer cells. In a human pancreatic adenocarcinoma cell line, inhibitors of V-ATPase and NHE1 have been shown to have an additive impact on intracellular pH and on thermosensitization; this suggests the desirability of evaluating joint administration of PPIs and cariporide in rodents.

Acute Intracellular Hyperacidification

A sufficiently large reduction in intracellular pH can promote apoptosis in cancer cells, could be used to achieve tumor-specific uptake or activation of certain drugs whose effects are pH-sensitive, and can potentiate the cytotoxic impact of local hyperthermia (˜42° C.) and TRAIL. This raises the interesting prospect that acute intracellular tumor acidification could be developed as a strategy for achieving rapid substantial tumor kill—perhaps when used in conjunction with other cytodisruptive measures whose activity is greater at acidic pH. This strategy has for potentiating the efficacy of concurrent local hyperthermia or chemotherapy.

An increase in tumor-specific acidification can be achieved if the rate of tumor glycolysis is maximized. Substrate availability for glycolysis is usually suboptimal in tumors, owing to the fact that, especially in aggressive tumors with high glycolytic capacity, tumor glucose levels tend to be relatively low owing to avid uptake of glucose for glycolysis and inefficient tumor perfusion. Hence, induced hyperglycemia tends to boost tumor glycolysis and decrease extracellular tumor pH by elevating tumor glucose levels; indeed, there are many reports that induced hyperglycemia tends to lower the extracellular pH of tumors, both in rodents and in human cancers in situ.

A further boost in glycolysis can be achieved by suppressing mitochondrial ATP generation; inhibitors or uncouplers of electron transport could be employed for this purpose. In many rodent and cell culture studies, an inhibitor of mitochondrial complex I, meta-iodobenzylguanidine, has been shown to amplify the depression of tumor intracellular pH achieved with hyperglycemia and/or proton extrusion inhibitors. This agent, in radioiodinated form, has been used in the treatment of neuroendocrine tumors—but the doses employed for this purpose are lower than those required for effective mitochondrial inhibition. A more practical and intriguing prospect in this regard is offered by the uncoupling agent dinitrophenol (DNP). DNP is mildly acidic, and its uncoupling activity is reported to be six-fold greater at pH 6.4 than at pH 7.4; thus, its impact might be amplified in acidified tumors.

Moreover, DNP was employed clinically in the 1930s as an anti-obesity agent. Although in excess it can give rise to lethal hyperthermia, it seems to be reasonably well tolerated in a daily dose of 3-5 mg/kg—a sufficient dose to raise the metabolic rate and promote substantial weight loss.

In human melanoma cell cultures, addition of DNP (0.1 mM) markedly potentiated the increase in glycolysis achieved with high glucose exposure. Sub-millimolar concentrations of DNP have been reported to slow proliferation, induce apoptosis, and exert a pro-oxidant effect in a human lung adenocarcinoma cell line; albeit the concentrations employed in this study are somewhat higher than would be systemically tolerable. These considerations suggest that it might be appropriate to examine the joint impact of hyperglycemia (achieved by sustained high-dose intravenous glucose infusion), PPIs, cariporide, and physiologically-tolerable concentrations of DNP on intracellular pH and survival of cancer cells in vitro and in rodents.

Such a strategy could also be assessed as an adjuvant to local hyperthermia or to administration of cytotoxic agents that are more active at acidic intracellular pH. If this strategy showed good efficacy in rodents, it could rapidly be translated to clinical application, as each of the drugs involved has already received substantial clinical evaluation and is known to be reasonably safe within defined dose levels. Owing to the requirement for hyperglycemia, this approach could only be used episodically. Conceivably, measures that raise extracellular tumor pH could be employed in the intervals between treatments.

Extracellular Hyperacidification Therapy:

Another approach to hyperacidification therapy would be to maximize the acidity of the tumor extracellular space (as with hyperglycemia and DNP, in the absence of proton pump inhibitors), with the intent of achieving tumor-selective delivery of cytotoxic drugs that are activated by acidity. In particular, nanoparticles that break down or fuse with cell membranes under mildly acidic conditions are being developed for selective drug delivery to acidified tumors. Concurrent administration of proton pump inhibitors would be expected ordinarily to impede optimal extracellular acidification, because the resulting suppression of intracellular pH would act as a brake on glycolysis, slowing the rate of lactate generation.

Unexpectedly, however, hyperacidification therapy that maximizes the acidity of the tumor extracellular space can be utilized longer term, for days to months, even in the presence of proton pump inhibitors. In this case, one can induce a metabolic acidosis while inhibiting efflux of protons from cancer cells, by concurrent administration of proton pump inhibitors, increasing cytotoxicity. This effect may be due to a decreased or reversed proton gradient from the intracellular to extracellular space. In some embodiments, this effect is enhanced by lactate administration, decreasing or reversing the lactate gradient from intracellularly to extracellularly.

Manipulation of the extracellular and/or intracellular pH of tumors has considerable utility in cancer therapy. At least five distinct strategies merit evaluation in this regard: (1) alkalizer therapy that increases the pH of the extracellular space; (2) proton pump inhibition that decreases the intracellular pH, while increasing the extracellular pH; (3) acute intracellular acidification that may be directly cytocidal or that potentiates the lethality of adjuvant measures; (4) acute extracellular acidification that enables tumorselective release of cytotoxic drugs from acid-labile nanoparticles; and (5) extracellular acidification—such as by using oral drugs taken over days to months that can induce a metabolic acidosis—combined with intracellular acidification that kills cancer cells directly or potentiates their sensitivity to other measures.

The extracellular acidity that characterizes most tumors—reflecting aerobic glycolysis sometimes induced by HIF-1 overactivity, as well as hypoxia in some tumor regions—tends to correlate negatively with cancer prognosis and is now known to be more than an epiphenomenon. Extracellular acidity can increase the invasive spread of cancer cells, while protecting them from immune attack and from the many cytotoxic agents that are mildly basic.

Feasible doses of safe alkalizing agents, such as sodium bicarbonate, can alleviate tumor acidification to some degree; in cancer-bearing mice, this strategy suppresses metastatic spread and improves response to chemotherapy. The extracellular acidity of tumors can also be corrected with proton pump-inhibitory drugs that are selectively activated in an acidic milieu. This approach has the ancillary advantage that it promotes the intracellular acidification of cancer cells; intracellular acidity tends to slow cellular proliferation while boosting apoptosis. Finally, inasmuch as intense intracellular acidification can be lethal, or can potentiate the lethality of other agents, acute hyperacidification therapies can be envisioned, in which measures that maximize cancer glycolysis (temporary induced hyperglycemia and possibly dinitrophenol) are employed concurrently with proton pump inhibitors. A variant approach would be to acutely amplify extracellular tumor acidity by maximizing tumor glycolysis in the absence of proton pump inhibitors, so as to induce selective uptake of concurrently administered drugs enclosed in acid-labile nanoparticles.

For PPIs, serum levels exceeding 10 mcg/ml inhibit vacuolar H+ ATPase in a dose dependent fashion. When combining a PPI with one or more other acid-efflux inhibitors (e.g., inhibitors of carbonic anhydrase, inhibitors of one or more Na+H+ exchanger, and/or inhibitors of one or more Cl−/HCO3 exchanger), one may be able to use lower doses of the PPI.

Doses found to be effective are from as low as 2.5 mg/kg, and are dose dependent. Doses used in Zollinger Ellison syndrome are up to 240 mg esopmerazole and 360 mg omeprazole, which are likely to produce serum levels exceeding 10 mcg/ml.

Na−H+ exchanger inhibitors include amiloride, HMA, DMA, cariporide, zoniporide, their derivatives and analogues, and the like.

Doses of amiloride found effective are as low as 0.3 mg/kg body weight per day and are dose dependent.

Cl−/HCO3 exchanger inhibitors that are effective include Trifolcin, DIDS, s3075, and levetiracetam, their derivatives and analogues, and the like.

Chloride-Bicarbonate Exchange Inhibitors

One example of a chloride-bicarbonate (Cl−/HCO3−) ion exchanger is Anion Exchanger 1 (AE1) or Band 3, which is a transport protein responsible for mediating the electroneutral exchange of chloride (Cl−) for bicarbonate (HCO3−) across plasma membranes. It is ubiquitous throughout vertebrates. In humans it is found in the erythrocyte (red blood cell) cell membrane and the basolateral surface of the alpha-intercalated cell (the acid secreting cell type) in the collecting duct of the kidney. Chloride-bicarbonate exchange inhibitors include 4,4′-diisothiocyanatostilbene-2,2′-disulfonate (DIDS), diphenylamine-2-carboxylate,

Proton-chloride (H+/Cl−) symporters

Cycloprodigiosin hydrochloride, a proton-chloride (H(+)/Cl(−)) symporter, induces apoptosis in human and rat hepatocellular cancer cell lines in vitro and inhibits the growth of hepatocellular carcinoma xenografts in nude mice.

The effects of cycloprodigiosin hydrochloride (cPrG-HCl) have been examined in, for example, liver cancer cell lines in vitro and in vivo. In an in vitro assay, cPrG-HCl inhibited the growth of 6 liver cancer cell lines (Huh-7, HCC-M, HCC-T, dRLh-84, and H-35, hepatocellular carcinoma; HepG2, hepatoblastoma) in a dose- and time-dependent manner. The 50% inhibitory concentrations (IC(50)) at 72 hours' treatment for liver cancer cell lines were 276 to 592 nmol/L, while that for isolated normal rat hepatocyte was 8.4 micromol/L. The cPrG-HCl treatment of Huh-7 cells induced apoptosis as confirmed by the appearance of a subG(1) population, intranucleosomal DNA fragmentation, and chromatin condensation. cPrG-HCl raised the pH of acidic organelles and lowered pHi (below pH 6.8). In addition, apoptosis in Huh-7 cells induced by cPrG-HCl was suppressed when the cells were cultured with imidazole, a cell-permeable base. In the in vivo assay, nude mice bearing subcutaneous xenografted Huh-7 cells received 2 weeks of treatment with cPrG-HCl (1 or 10 mg/kg/d) subcutaneously. This treatment significantly inhibited tumor growth compared with the control after 8 days. The control mice were treated with 1% dimethylsulfoxide (DMSO) in saline (vehicle). A histopathological examination using the terminal deoxynucleotidyl transferase mediated dUTP biotin nick end labeling (TUNEL) method showed apoptosis in the treated tumor cells. No pathological changes were observed in any organs, and the serum alanine transaminase levels remained within normal limits. cPrG-HCl may be useful for the treatment of hepatocellular carcinoma.

Proton Transport Inhibitors

A proton [H+]-related mechanism at least partly underlies the initiation and progression of the process by which cancer cells, regardless of their origin and genetics, have an energetic and homeostatic disturbance of their metabolism that differs from normal tissues: an aberrant regulation of hydrogen ion dynamics leading to a reversal of the normal pH gradient, from intracellularly to extracellularly, in cancer cells and tissues (ApHi to ApHe). This abnormality of the relationship between intracellular and extracellular proton dynamics, an important differential feature of cancer, implicates issues such as pathogenesis, cancer cell metabolism, multiple drug resistance, neovascularization, metastasis mechanisms, selective apoptosis, chemotherapy mechanisms, and spontaneous regression of cancer. This reversed proton gradient is driven by a series of proton export mechanisms that underlie the initiation and progression of cancer processes. Therapeutic targeting of transporters active in cancer cells can be selective for malignancy, providing a pathway to more effective, less toxic therapies for malignancies.

The induction and/or maintenance of intracellular alkalinization and its subsquent extracellular, interstitial acidosis on intratumoral dynamics have been repeatedly implicated as playing a role both in cell transformation as well as in the active progression and maintenance of the neoplastic process. Indeed, this specific and pathological reversal of the pH gradient in cancer cells and tissues (ΔpH to ΔpHe) compared to the normal tissue pH gradient is considered to be one of the main characteristics defining the molecular energetics of tumors, regardless of their pathology and genetic origins. Aberrant regulation of hydrogen ion dynamics leading to this reversed proton gradient is driven by a series of proton export mechanisms that underlie the initiation and progression of the neoplastic process.

While the hyperactivity of the Na+/H+ exchanger isoform 1 (NHE1) is an important component in the up-regulation of proton extrusion and in its secondary activation of cell transformation, proliferation, motility, and invasion of cancer cells derived from a wide array of tissues, it is not the only plasma membrane-bound membrane transporter/enzyme responsible for cytosolic alkalinization of the tumor cell and acidification of the extracellular space. Additionally, the vacuolar H+-ATPases, the H+/Cl-symporter, the monocarboxylate transporter (MCT, mainly MCT1) (also known as the lactate-proton symporter), the Na+-dependent Cl−/HCO3-exchanger, ATP synthase, and the Na+/K+-ATPase can also play an important role in proton extrusion, pHi abnormalities, and tumor interstitial acidification in different human malignancies.

Studies on tumor microenvironment pH have shown evidence that some carbonic anhydrase (CAs) isozymes, mainly CAII, CAIX and CAXII, are overexpressed in various types of human tumors, an up-regulation that is inversely related to prognosis; and they also make a significant contribution to the extracellular acidity, which is one of the main functional hallmarks of invasive cancer, therefore representing promising targets for novel anticancer therapies. These findings suggest that the targeting of proton transporters may be used to trigger selective cancer cell death through the induction of low pHi-mediated apoptosis. Tumor cell proliferation is abolished through the concerted inhibition of NHE1 and Cl−/HCO3− exchangers. Similarly, while Cl−/HCO3− exchanger inhibition alone may be insufficient to induce apoptosis in breast cancer cells, the simultaneous inhibition of the NHE1 and H+-ATPase induces apoptosis through their concurrent effects on lowering pHi.

The failure of tumor cells to die following chemotherapeutic treatment also often appears to be highly dependent on their resistance to undergo intracellular acidification, a low intracellular pHi homeostatic situation that is apparently necessary as a prior and early condition that allows cancer cells to engage in a tumor-specific apoptotic process. One aim is to target this specific aspect of cancer cell metabolism based on the H+-dependent thermodynamic advantages that malignant cells possess as compared to their normal counterparts. The exploitation of such differences in selective cancer therapeutics as chemotherapy adjuvants is a possible strategy that could decrease chemotherapy dosages while at the same time increasing therapeutic specificity and effectiveness regardless of tumor type and origin.

Thermodynamic Strategy of Cancer Cells

During and after neoplastic transformation, a thermodynamically advantageous reversal of the previously normal situation takes place, namely, the reversal of the transmembrane H+-gradient (alkaline inside, acidic outside), a specific feature described only in malignant disease. The main mechanism of this reversal is an intracellular alkalinization mediated by the systemic extrusion of H+ by the different proton transporters (PTs) described above, while the chloride bicarbonate exchanger brings in a bicarbonate anion exchange for a chloride anion to neutralize protons inside the cell. However, because not all cancer cells necessarily have the same transporters elevated at the same time, it appears that in order to maintain the abnormal cellular alkalinity, when one transporter is inhibited others can become up-regulated. The observed consequences of this initial cellular acid-base energetic change demonstrates that one of the main purposes of the biochemistry and metabolism of the concerted, dynamic, energetic defensive systems of cancer cells and tissues is to have the different transmembrane proton transport mechanisms working, at least when required by damaging microenvironmental conditions, to first create and then maintain a cascade of electrochemical changes and events leading to tumor development, local growth and invasion, the activation of the metastatic process and, simultaneously, resistance to treatment.

These mechanisms driven by the loss of the normal acid-base homeostatic balance of the cell (initial and specific cause for cell transformation) are: A) Maintenance of a normal to elevated intracellular pH even under the circumstances of the metabolic microenvironment of cancer cells (interstitial acidosis, lack of blood supply, low O2 conditions) in order to protect themselves from low pHi-mediated apoptosis, at the same time that they initiate an unregulated and thermodynamically beneficial proliferative and invasive state; B) The establishment of a self-defensive, anti-apoptotic strategy mediated through different anti-acidifying mechanisms such as hyperactivity of the different membrane-bound proton extrusion transporters, inactivation of Bcl-2, Bcl-xl, and destabilization of p53; C) These concerted dynamic changes are based upon the advantageous utilization of a H+-gradient reversal function as an anti-chemotherapeutic shield involved in multiple drug resistance (MDR) and in the development of newly resistant subpopulations of tumor cells; D) The above mechanisms lead to secondary acidification of the interstitial component of tumors, in either low or normal O2 conditions, which is key to the onset of local invasion and to the activation and maintenance of the metastatic process by increasing the expression of a wide array of positive angiogenic factors (e.g. HIF-1, VEGF), while the extracellular acidification of tumors creates even further resistance to chemotherapy, radiation-induced apoptosis and hyperthermia.

(A) Self-protection against the caustic extracellular tumor microenvironment. While non-transformed cells and tissues die under conditions of extracellular acidosis, the multipletransporter strategy allows malignant cells of diverse origins to defend themselves from any acidic and/or therapeutic and/or apoptotic attack by taking advantage of a concerted system of membrane-bound ionic transporters whose main role is to extrude hydrogen ions from the cell. This allows transformed cells of all genetic origins to first survive and then multiply under these extremely difficult environmental circumstances. This tumor-specific metabolic condition suggests a possible therapeutic solution: it is this same highly pathological and specific pH gradient reversal of all cancer cells and tumors which becomes the key factor that offers the opportunity to target it as one of the few, if not the only truly differential characteristic that separates all malignant tissues from all normal ones. This would be to attempt to selectively induce a cancer cell self-poisoning through diverse low pHi-related therapeutic measures.

Because no cancer cell can survive for long with pHi conditions below a certain acid threshold, successful therapeutic interventions targeting this H+-mediated gradient reversal through the concerted utilization of proton transport inhibitors (PTIs) of the different families can become a key therapeutic strategy to selectively trigger the apoptotic process in malignant cells and tissues.

(B) pHi and selective apoptosis in cancer. Low pHi can induce apoptosis. A series of studies using different chemotherapeutic substances in a variety of tumor cells have reported that cytosolic acidification is a very early event in the onset of malignant cell apoptosis. The induction of an intracellular acid environment has been reported to trigger the onset of apoptosis of leukemic cells by upregulating the expression of Bax protein expression, which is pro-apoptotic. This seems to be mediated by the activation of interleukin-1β-converting enzyme (ICE/caspase-1) or the apoptosis-effector protease CPP32 (Caspase-3), irreversibly leading to acid stress-induced apoptosis, and thus to the control of cell proliferation and arrest of tumor growth. Inhibition of the NHE1 plays a fundamental role in paclitaxel-induced apoptosis of breast cancer cells and this is synergistically potentiated by inhibition of the NHE1 with the amiloride analog, 5-(N,N-dimethyl)amiloride DMA), while the recently developed, potent inhibitor of the NHE1, cariporide (HOE-642) seems to induce a similar effect. Because NHE1 inhibition reduces transformed cell pHi well below parental cell values, and this intracellular pH decrease does not show a significant effect on normal cells, this indicates a certain degree of therapeutic selectivity and specificity for at least certain NHE1 inhibitors in malignancy (22), even more so because proton transporters are differently expressed in normal and tumor tissues.

Results in different kinds of leukemic cells with the potent amiloride derivative inhibitor of NHE1,5-(N,N-hexamethylene)-amiloride (HMA), which reduces the intracellular pH well below the acid-base survival threshold, shows that inducing a low pHi-mediated apoptosis is a selective therapeutic modality for many different cancer cells and tissues. Similarly, to inhibit the MCT, lonidamine and cinnamate have been used. The compound a-cyano-4-hydroxycinnamate (4-CIN) and its pharmacologically active derivatives also inhibit the MCT. Recently, AstraZeneca developed agents that inhibit MCT in the nanomolar range and are more specific. MCT levels have been found to be high in neuroblastoma cells and in melanoma cells exposed to a low pHe. In neuroblastoma cells, the gene for the MCT (SLC16A1) is amplified, and not only does cell death occur as a function of pHe in vitro, but the correlation between high levels of MCT and poor prognosis is found in children with neuroblastoma, a pediatric malignancy with a very high mortality. Parallel results are obtained when other proton transporters, such as CAIX, are considered.

(C) pHi, pHe and MDR. A direct cause-effect relationship among the degree of MDR and the elevation of tumor pHi has characterized the dynamic interrelationships between cell pHi and MDR.

High pHi, mediated either by overexpression/activity of the NHE1 and/or other proton-extruding mechanisms such as VATPases, MCTs and carbonic anhydrases (CAs) have been found to be responsible for cisplatin resistance and, similarly, to contribute to the onset and/or maintenance of MDR, so protecting against tumor cell death from anticancer drugs. Furthermore, drugs such as adriamycin, cisplatinum, paclitaxel and camptothecin have been shown to be unable to induce apoptosis under non-acidified cellular conditions and, indeed, resistance to several anticancer drugs such as camptothecin, vinblastine, adriamycin and etoposide has been shown to be dependent on overexpression of different proton transporters and/or intracellular alkalinization. Recently, third generation camptothecin analoges have been developed that are more active at low pH. This design should lead to more selectivity and less toxicity of this chemotherapeutic agent. In this context, specific H+-ATPase inhibitors, such as bafilomycin A1, salkylihalamide, lobatamides and oximidines have been also considered as potential anticancer agents and MDR-reversal agents, in a similar way to CAIX inhibitors such as acetazolamide.

The fact that cells with an active MDR transporter show cytoplasmatic alkalinization has led some authors to conclude that P-glycoprotein can be mainly considered as a proton extrusion pump. However, Pglycoprotein (P-gp) activity is stimulated by interstitial acidification secondary to the abnormal H+-dynamics of cancer tissues and, indeed, the therapeutic failure to induce cytoplasmic acidification has been proposed as the main underlying factor for MDR because of resistance to the induction of therapeutic apoptosis in both normal or slightly alkaline and highly alkaline cancer cells. Thus, in many instances it seems that MDR can be attributed to the failure to induce intracellular acidification by compounds such as chloroquine, imidazol, glutathione, apart from overexpression/activity of proton transporters. Finally, the MDR-promoting effects of the Bcl-2 family of proteins, as well as a dysfunctional p53, which also contribute to pro-carcinogenic and antiapoptotic effects, have also been shown to be dependent on their ability to maintain a sufficiently elevated intracellular cell pH in order to avoid therapeutic apoptosis.

All these findings are further corroborated by the fact that a large variety of MDR modifiers known to be able to revert resistance to chemotherapeutic drugs (e.g. verapamil, amiodarone, bafilomyicin A1, cyclosporine A, tamoxifen, DIDS, nigericin and edelfosine), have all been reported to exert their cellular effects, at least in part, through pHi-acidifying mechanisms. A decrease in pHi has been shown to sensitize cancer cells of diverse origins to apoptosis, chemotherapy and hyperthermia, or to induce apoptosis by themselves. Indeed, a reversal of MDR can be obtained by the pH-lowering effects of amiloride and/or its analogs in a variety of situations. In summary, a selective and concerted role for PTIs as chemotherapy adjuvants in MDR, as well as selective anticancer agents on their own, could well be a very successful strategy. This would decrease chemotherapy dosages and toxicity while at the same time increase therapeutic specificity and effectiveness regardless of tumor type and origin.

(D) Relationships between hydrogen ion dynamics, malignant neovascularization and the metastatic process. Neovascular growth and metastasis are direct consequences of the hostile environment of low extracellular pH as well as of low interstitial p02. Indeed, the high pHi-low pHe-proton gradient reversal factor by itself can induce vascular endothelial growth factor (VEGF) production, without the need of any other structural intermediate such as hypoxiainducible factor 1 (HIF-1), and which, at the same time, can be inhibited by lowering intracellular pH and/or collapsing the proton gradient reversal with amiloride. This H+-gradient reversal has also been shown to induce not only the expression of VEGF but also of insulin-like growth factor 1 receptor (IGF1R), platelet-derived growth factor β-receptor, interleukin 8 and metalloproteases.

Recent research trends have mainly focused on tumoral hypoxia as a source of VEGF and has emphasized its role in the metastatic process. However, the proven role of relative hypoxia as a direct etiological factor in cell malignant transformation, as initially proposed by Warburg—what was previously called by us the Warburg-Goldblatt effect—still needs to be taken into account as an etiological factor of its own in cell malignant transformation in a manner similar to high pHi (low intracellular H+-concentration functionally mimicking low pO2, or para-hypoxia).

Intracellular signalling factors and mechanisms targeting pHi and the Na+/H+ exchanger in apoptosis: Factors that induce apoptosis through intracellular acidification as its common final pathway. This integrated and homeostatic pH-related perspective can help to foretell pro-apoptotic and anti-apoptotic factors in order to find synergistic therapies and potential antagonisms (MDR) in anticancer treatment.

The role of NHE1 activity and/or an abnormally increased pHi in stimulating different steps of the metastatic process is significant. The activity of a significant number of proangiogenic factors and oncogenes has been shown to be directly related to NHE1 expression, while, on the contrary, a wide array of antiangiogenic drugs inhibit the NHE1. In addition, other pro-metastatic mechanisms are sensitive to inhibitors of NHE1 activity, such as the urokinase-type plasminogen activator (μPA), matrix metalloproteinase (MMP-9) and the cathepsin Bdependent activation of MMP-2 and MMP-9. It has long been known that amiloride can achieve a complete in vivo antimetastatic effect in different transplanted tumors.

Thus, amiloride and, mainly, its more potent derivatives, have been increasingly considered as a novel, adjuvant and neoadjuvant treatment for cancer in order to reduce tumor growth and increase patient survival.

Role of Tumoral pHe in Invasion and Extracellular Protease Action

Multiple studies have strongly supported a pathogenic role of the acidic interstitial pHe of tumors by giving a selective advantage for tumor progression and metastasis. It has been shown to drive large changes in gene expression independently of hypoxia and has also been associated with tumor progression by impacting multiple processes including increased invasion and metastasis. This can occur directly or through the alteration of the extracellular matrix (ECM) compartment through up-regulation of protease secretion/activation and in an altered tumor-stromal interaction via an inverse stimulation of pro-angiogenic factors paired with impaired immune functions.

Proteolytic ECM remodeling is a prerequisite for the invasive process. Indeed, the proteolytic breakdown of proteins of the ECM is one of the first steps in invasion in primary cancer lesions. During invasion, cancer cells use secreted, surface-localized and intracellular cathepsins, serine proteases and MMPs to proteolytically cleave, remove and remodel different types of ECM substrates at the cell surface, including collagens, laminins vitronectin, and fibronectin (61). While tumor-driven extracellular acidification of the tumor pericellular space can directly drive the destruction of the surrounding normal limitrophic tissue, a large body of work has demonstrated that the acid pHe of tumors can also indirectly drive ECM proteolysis by increasing protease production and secretion of the active forms of the cathepsin family of proteases, such as cathepsin D, cathepsin B, cathepsin L, MMP-9, and MMP-2. There is evidence demonstrating that NHE1 and its associated extracellular acidification is necessary for the (i) cathepsin B-dependent ECM proteolytic activity and invasion of breast cancer cells in which the ECM receptor, CD44, was activated by hyaluronan, and (ii) MMP-9 activation and invasion in non-small lung cancer cells in which alpha1-adrenergic receptor was stimulated by phenylephrine. Interestingly, one study observed that the low pHe-driven activation of MMP-9 and MMP-2 was dependent on the up-stream activation of cathepsin B and all three proteases were located on small vesicles shed from the tumor cell. This increased secretion and activity of proteases is congruent with the known increased invasive capacity at acid pHe.

The acid pHe of tumors has also been shown to alter the interactions between tumor cells and the cells of both the stromal compartment and the immune antitumoral defense system. On the one hand, acidic pHe has been demonstrated to increase the expression and secretion of angiogenesis promoting and metastatic factors such as VEGF and interleukin-8 (IL8). On the other hand, there is evidence that the acid component of the tumor microenvironment also directly reduces/impairs the function of the antitumoral immune system, thus contributing to the known in vivo immunosuppression. Exposure to increasingly acidic pHe has been shown to reduce tumor cell-induced cytolytic activity of lymphokine-activated killer (LAK) cells, to play a role in down-regulating cytolytic activity of tumor-infiltrating lymphocytes with natural-killer (NK) phenotype and to inhibit the non-major histocompatibility complex (MHC)-restricted cytotoxicity of immunocompetent effector cells.

Altogether, these studies indicate that an acidic tumoral interstitial pHe promotes invasion and metastasis by a reciprocal mechanism involving acidity-induced upregulation of proteolytic enzymes and pro-angiogenic substances together with an acidity-induced down-regulation or impairment of the organisms antitumoral immune defense. One consequence of this situation is that treatment strategies should be aimed by all means at collapsing the intracellular/extracellular H+-gradient inhibiting PTs in order to increase selective intracellular acidification and apoptosis, plus (an apparent paradox) alkalinizing the tumor interstitial space by blocking the mechanisms driving its acidification, while avoiding any therapy that could involve deliberate tumor extracellular acidification.

H+-related Mechanisms in the Spontaneous Regression of Cancer (SRC)

The favorable influence of acidification on complete cancer regression in a wide array of transplanted animal tumors has also been recognized over the years. Severe metabolic acidosis induced by some surgical procedures, such as ureterosigmoidostomy, infections and febrile processes, was initially considered to be the main and ultimate underlying mechanism behind some spontaneous regressions of malignant tumors in animals and human beings. Recently, a graded metabolic acidosis associated with mild renal failure was claimed to reduce, and even reverse, the rates of tumor growth and invasion in cancer patients.

Etiopathogenesis-based therapeutics. PTIs as potential and selective anticancer agents in the treatment of human malignant diseases.

While proton research in cancer cannot yet be considered to be within the mainstream of modern oncology research, the increasing evidence accumulated during the last few years points to the fact that the dynamics and metabolism of the hydrogen ion are becoming a subject of growing interest as a potential key target in selective therapeutic intervention in leukemias, solid tumors and other chronic degenerative diseases.

The H+-related perspective briefly reviewed here suggests a new paradigm able to encompass an enormous and scattered bulk of information in the main areas of cancer research as has been advanced in recent reports. An advantage of such a unified basic approach is the possibility of integrating what previously were considered to be non-interrelated areas of research, in order to translate their data and interrelationships into a more complete and encompassing integrated synthesis and, from there, into clinical therapeutics.

Besides improving our basic understanding, this paradigm could stimulate further integrations between biochemical and metabolic cancer research to molecular biology and cancer immunity. The latter relationship is exemplified by the fact that therapeutic cell death in lymphomas depends on NHE1 inhibition by IGM-mediated cell death through intracellular acidification. The negative effects of tumoral interstitial acidification in reducing cellular immunity are also well known.

In spite of the fact that transmembrane H+ gradient reversal appears to be the single most differential molecular characteristic setting apart cancer cells and tissues from normal ones, this feature still remains to be exploited in the treatment of human cancer. Taking into account the theoretical background available and the results of different cell studies, animal experimentation and occasional reports in cancer patients that justify this H+-dependent approach, the situation is difficult to understand. This is probably due to the fact that the most active agents that could revert the abnormal H+-gradient situation, such as the potent amiloride-derivatives like HMA, cariporide, and zoniporide, are still waiting to be included in pre-clinical or clinical trials. In this respect, all the available data would suggest the importance of undertaking prospective studies in different human malignancies in order to test the therapeutic, pro-apoptotic, antimetastatic and MDR-overcoming concerted effects of PTI drugs, from the more potent derivatives of the amiloride series to other NHE1, V-ATPase, MCT1, HCO3−/Cl− exchangers and CA inhibitors.

In summary, the main bulk of both seminal and emerging data briefly reviewed in this contribution leads to three consistent conclusions: i) Cell alkalinization constitutes an initial and fundamental event in the transformation process of normal cells and tissues regardless of their origin; ii) The overexpression/activation of a number of membrane-bound proton transporters plays a positive key role in later neoplastic and metastatic progression and a negative role in the host defense mechanisms (e.g. antiangiogenesis, spontaneous regression, therapeutics) by severely disrupting intracellular/extracellular proton gradients and inducing a subsequent alteration in cell thermodynamics; iii) Targeted inhibition of the different proton transporters is a promising area in seeking selective anticancer treatments useful in preventing, retarding, or counteracting the neoplastic process at different levels. The concerted utilization of PTIs, alone or in combination with other forms of chemotherapy, may prove fundamental in primary, adjuvant and/or neoadjuvant treatment of different solid tumors in humans, as well as in the overcoming of MDR.

Agents that can lower intracellular pH include somatostatin and somatostatin derivatives that act as G protein-coupled receptor agonists; somatostatin receptors; nigericin, a proton ionophore; bafilomycin compounds, including bafilomycin A-1; DIDS (4,4-diisothicyanatostilbene-2,2-disulfonic acid); EIPA (5-(N-ethyl-N-isopropyl)-amiloride; and other amiloride-based compounds.

The G protein-coupled receptor agonist somatostatin (SST) induces apoptosis in MCF-7 human breast cancer cells. This is associated with induction of wild-type p53, Bax, and an acidic endonuclease. This cytotoxic signaling is mediated via membrane-associated SHP-1 and is dependent on decrease in intracellular pH (pHi) to 6.5. Clamping of pHi at 7.25 by the proton-ionophore nigericin abolishes SST-signaled apoptosis without affecting its ability to regulate SHP-1, p53, and Bax. Apoptosis can be induced by nigericin clamping of pHi to 6.5. Such acidification-induced apoptosis may possibly not occur at pHi<6.0 or >6.7. pHi-dependent apoptosis is associated with the translocation of SHP-1 to the membrane, enhanced in cells overexpressing SHP-1, and can be abolished by its inactive mutant SHP-1C455S. Acidification caused by inhibition of Na1/H1 exchanger and H1 ATPase (pHi 5 6.55 and 6.65, respectively) can also trigger apoptosis. The effect of concurrent inhibition of Na1/H1 exchanger and H1-ATPase on pHi and apoptosis can be comparable with that of SST.

Acidification-induced, SHP-1-dependent apoptosis occurs in breast cancer cell lines in which SST is cytotoxic (MCF-7 and T47D) or not (MDA-MB-231). It appears that: (a) SST-induced SHP-1-dependent acidification occurs subsequent to or independent of the induction of p53 and Bax; (b) SST-induced intracellular acidification may arise due to inhibition of Na1/H1 exchanger and H1-ATPase; and (c) SHP-1 may be important, or even necessary, for agonist-induced acidification and also for the execution of acidification-dependent apoptosis. Combined targeting of SHP-1 and intracellular acidification can contribute to strategies of anticancer therapy bypassing the need for receptor-mediated signaling.

Vacuolar-type H+-ATPase (V-ATPase) is a highly conserved evolutionarily ancient enzyme with remarkably diverse functions in eukaryotic organisms. V-ATPases acidify a wide array of intracellular organelles and pump protons across the plasma membranes of numerous cell types. V-ATPases couple the energy of ATP hydrolysis to proton transport across intracellular and plasma membranes of eukaryotic cells.

Proton Pump

In mitochondria, reducing equivalents provided by electron transfer or photosynthesis power this translocation of protons. For example, the translocation of protons by cytochrome c oxidase is powered by reducing equivalents provided by reduced cytochrome c. In the plasma membrane proton ATPase and in the ATPase proton pumps of other cellular membranes, ATP itself powers this transport.

The FoFI ATP synthase of mitochondria, in contrast, usually conduct protons from high to low concentration across the membrane while drawing energy from this flow to synthesize ATP. To allow the passage of protons a proton channel temporarily opens in the inner membrane.

The gastric hydrogen potassium ATPase or H+/K+ ATPase is the proton pump of the stomach which is primarily responsible for the acidification of the stomach contents.

In some embodiments, proton pump inhibitors are given in an inactive form. The inactive form can be neutrally charged and lipophilic and readily crosses cell membranes into intracellular compartments (like the parietal cell canaliculus) that have acidic environments. In an acid environment, the inactive drug is protonated and rearranges into its active form. The active form will covalently and irreversibly bind to the gastric proton pump, deactivating it.

Potassium-Competitive Acid Blockers (P-CABs)

Potassium-competitive inhibitors reversibly block the potassium binding site of the proton pump. Soraprazan and revaprazan block H+ secretion much more quickly than classical PPIs (within a half-hour). The development of soraprazan, however, was discontinued in 2007.

Examples of Proton Pump Inhibitors

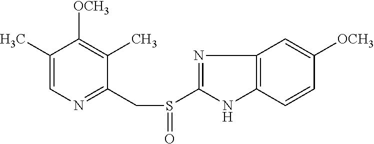 [SMILES: COc1ccc2[nH]c(S(=O)Cc3ncc(C)c(OC)c3C)nc2c1]

The proton pump inhibitor omeprazole.

Clinically used proton pump inhibitors:

Omeprazole (brand names: Losec, Prilosec, Zegerid, ocid, Lomac, Omepral, Omez)

Lansoprazole (brand names: Prevacid, Zoton, Inhibitol, Levant, Lupizole)

Dexlansoprazole (brand name: Kapidex, Dexilant)

Esomeprazole (brand names: Nexium, Esotrex)

Pantoprazole (brand names: Protonix, Somac, Pantoloc, Pantozol, Zurcal, Zentro, Pan)

Rabeprazole (brand names: Zechin, Rabecid, Nzole-D, (NEHAL PHARMA Pvt. Ltd.), AcipHex, Pariet, Rabeloc. Dorafem: combination with domperidone.

Bafilomycin A1 as an inhibitor of the proton pump V-ATPase

Proton pump inhibitors inhibit cell growth and induce apoptosis in human hepatoblastoma. Ordinarily, a vacuolar-type proton pump (V-ATPase) maintains an intracellular acid microenvironment in lysosome, endosome, and other endomembrane systems. Cancer cells overexpress V-ATPase compared with normal cells, and disturbances of the acid environment are thought to significantly impact the cancer cell infiltration and growth. Bafilomycin A1 (Baf-A1) is a specific proton-pump inhibitor (PPI) of V-ATPase. Neoplastic cells are reportedly more sensitive to Baf-A1 than normal cells, and the difference between the susceptibility to Baf-A1 in normal cells and that in cancer cells may become a target in the cancer therapy. With this in mind, we used cells of hepatoblastoma, the cancer type accounting for 80% of all childhood liver cancers, to investigate the effects of Baf-A I as an inducer of cancer cell apoptosis and inhibitor of cancer cell reproduction

Electron microscopy shows significant morphological change of hepatoblastoma cells of a Baf-A1-treated group compared with hepatoblastoma cells of a Baf-A1-free group. In one study, the rate of the apoptosis increased, and cell reproduction was inhibited. Moreover, the analysis of hepatoblastoma cells using the gene Chip gene expression analysis arrays showed that three of the 27 V-ATPase-related transcripts (ATP6VOD₂, ATP6V1B1, and ATP6V0A1) were more weakly expressed in Baf-A1-treated cells than in Baf-A1-free cells. In normal human hepatic cells, the inhibition of cell growth of the Baf-A1-treated cells is negligible compared to that of the cells without Baf-A1 treatment. The result of apoptotic cell detection by morphological observations and flow cytometry reveals that Baf-A1 inhibits hepatoblastoma cellular reproduction by inducing apoptosis. On the other hand, the Baf-A1-conferred inhibition of cell growth is negligible in normal human hepatocytes

Thus, the V-ATPase inhibitor Baf-A1 has been proven to selectively inhibit the reproduction and induce the apoptosis of hepatoblastoma cells without adversely influencing normal hepatic cells. V-ATPase inhibitors may therefore be used as therapeutic agents for hepatoblastoma and other cancers. Given that three V-ATPase-related genes (ATP6V0D2, ATP6V1B1, and ATP6V0A1) were more weakly expressed in the hepatoblastoma cells of the Baf-A1-treated group than in the Baf-A1-free cells, further drug development targeting V-ATPase gene of hepatoblastomas is contemplated.

Na+-dependent Cl−/HCO3− exchange

The anticonvulsant levetiracetam is an example of an inhibitor of Na+-dependent Cl−/HCO3− exchange. The antiepileptic mechanisms of levetiracetam (LEV) are not fully characterized, although attempts have been made to uncover the effects of LEV on inhibitory or excitatory pathways. For instance, GABAergic currents are not influenced by LEV in paired-pulse studies of field potentials. Yet, LEV inhibits blockade of bicuculline on GABAergic currents and, by this, decreases bicuculline-induced neuronal hyperexcitability. LEV can oppose the effects of negative modulators of GABAA-mediated currents. However, the mechanism underlying of LEV's modulation of GABAergic currents remains obscure.

LEV also apparently fails to block voltage-gated Na+ and low-voltage-activated Ca2+ currents as well as NMDA receptors. Also, there is apparently incomplete inhibition of voltage-operated K+ currents and N-type Ca2+ channels of hippocampal CA1 neurons whose high-voltage-activated calcium currents are reduced by about 20%. Although the latter channels are overexpressed after kindling, their contribution to the genesis of epileptic potentials is still unclear. Kindling-induced alterations in gene expression in temporal lobe of rats are modified by LEV. Thus, LEV seems to affect synchronization of epileptiform events rather than affecting synaptic transmission.

Acidification of neuronal tissue reduces neuronal excitability, whereas alkalinization increases it. Changes of intracellular pH (pHi) are used to reduce epileptiform activity in epileptic model systems.

Hippocampal slices treated with 4-aminopyridine (4-AP) respond to a lowering of neuronal pHi by decreased bioelectric activity. Clinically relevant concentrations of LEV affect Na+-dependent Cl−/HCO3− exchange, which is of biological significance for pHi regulation of hippocampal neurons.

Epileptiform activity of CA3 neurons has been induced by 4-AP (50 μM) added to a CO2/HCO3−-buffered solution to reach a stable state of hyperexcitation for hours, characterized by epileptiform bursts and spontaneous GABAergic hyperpolarizations.

To analyze pHi changes, hippocampal slices have been loaded with 0.5-1.0 2′,7-bis(2-carboxyethyl)-5(6)-carboxyfluorescein-acetoxymethyl ester (BCECF-AM, Molecular Probes, Leiden, Netherlands) for 3 min in preincubation saline. Measurements of individual CA3 somata (identified by their apical dendrites) were carried out with a ×60 water-immersion objective (Olympus), which dipped into the fluid of the recording chamber.

To examine whether LEV induces a neuronal acidification, BCECF-AM-loaded slices and observed pH-dependent fluorescent signal from single neurons or expanded regions of the stratum pyramidale have been employed by other researchers. In CO2/HCO3−-buffered solution, steady-state pHi measured in neuronal somata was 7.05±0.16 (n=11). Application of LEV (50 μM) lowered pHi in eight of 11 neurons. After a stable steady state had been reached (after 5-18 min), the mean acidification during LEV amounted to 0.14±0.09 pH units (n=11). This value increased to 0.19±0.07 pH units (n=8) if the three nonresponding neurons were excluded. A further increase of LEV concentration to 100 μM was without additional effect (n=4). Small shifts of steady-state pHi were also observed upon 10 μM LEV (n=3); 1 μM LEV was without any effect on pHi (n=4). Thus, the concentration of LEV relevant for changes of pHi in slice experiments ranged between 10 and 50 μM. Therefore, further experiments were carried out with 50 μM LEV. Washout of LEV increased pHi within 20-32 min to values being slightly decreased compared to the control phase before LEV.

Effect of LEV on the neuronal steady-state pHi. (a) pHi of a BCECF-AM-loaded CA3 neuron was reversibly lowered by 50 μM LEV in CO2/HCO3−-buffered solution. (b)

Effect of LEV (50 μM) on pHi measured in the stratum pyramidale (size of each region was approximately 200×40 μm2). In the absence of extracellular Na⁺ (Na+-free) and in the absence of extracellular CO2/HCO3−(CO2/HCO3−-free), pHi remained unchanged while LEV decreased pHi in CO2/HCO3−-buffered solution.

In CO2/HCO3−-buffered solution application of LEV (50 μM) decreased pHi of larger regions by 0.11±0.04 pH units (n=5, FIG. 2b). In the nominal absence of extracellular CO2/HCO3− and in the presence of 4-(2-hydroxyethyl)-1-piperazineethanesulfonic (HEPES) (pH 7.4), the decrease of pHi upon LEV was abolished (n=5, FIG. 2b). LEV can affect steady-state pHi in Na+-free CO2/HCO3−-buffered solution. Withdrawal of Na+ from the bath solution decreases pHi in the stratum pyramidale by 0.5±0.14 pH units to a new steady state. This value is not likely further influenced by LEV (50 μM, n=5). These experiments show that LEV-mediated lowering of the steady-state pHi depends on inwardly directed gradients of both HCO3− and Na+.

pHi Regulation in CO2/HCO3−-Free, HEPES-Buffered Solution

In the next set of experiments, researchers used the ammonium prepulse technique to study the effects of LEV on pHi regulation. Slices loaded with BCECF-AM in CO2/HCO3−-buffered solution were equilibrated with HEPES-buffered solution for at least 20 min before the first ammonium prepulse (20 mM, 3 min) was applied. Under control conditions, an ammonium prepulse typically resulted in an alkalinization phase, followed by an acidification upon NH4Cl washout, and a final phase of On recovery due to proton extrusion. To examine the effects of LEV (50 μM), the drug was washed in 15 min before the second ammonium prepulse. Similar to its lacking influence on steady-state pHi, LEV did not change the ammonium prepulse-induced pHi regulation in CO2/HCO3-free solution. pHi recovery rate was 1.61±0.59×10-2 pH units/min and 1.78±0.36×10-2 pH units/min in the absence and presence of LEV, respectively (n=5, P=0.43). These findings indicate that it is unlikely that LEV at this concentration inhibits HCO3-independent acid extrusion, such as Na+/H+ exchange.

pHi Regulation in CO2/HCO3−-Buffered Solution

In the presence of extracellular CO2/HCO3− regulation of pHi is affected by LEV (50 μM, pretreatment 15 min). Within the stratum pyramidale, LEV significantly decreases pHi recovery from 0.85±0.10×10-2 pH units/min to 0.26±0.16×10-2 pH units/min (P=0.005, n=5, FIG. 3b1). Also, in individual neuronal somata, the same concentration of LEV lowers pHi recovery from 1.27±0.44×10-2 pH units/min to 0.68±0.25×10-2 pHi units/min (P=0.003, n=4). After pHi recovery subsequent to the first ammonium prepulse (control), preincubation with LEV lowered the steady-state pHi to a value of 6.82. The second ammonium prepulse increased pHi by ca. 0.2 pH units, thus reaching the value it had adopted upon NH4Cl application in the absence of LEV. After the acidotic peak, the pHi recovery rate was clearly slowed. Washout of LEV then initiated the increase of pHi. It should be pointed out that LEV did not inhibit pHi recovery of two neurons, in which steady-state pHi did not fall during preincubation with LEV. These findings point to an LEV-mediated inhibition of transmembrane HCO3− influx, which is used by hippocampal neurons to regulate pHi after an acid load.

Cl−/HCO3− Exchange

LEV has been studied for its effect on Na+-independent Cl−/HCO3− exchange, as this electroneutral antiport is also involved in the adjustment of steady-state pHi and pHi regulation of hippocampal neurons. A 4,4′-diisothiocyanato-stilbene-2,2′-disulfonic acid (DIDS)-sensitive alkalinization upon removal of extracellular Cl− is usually believed to be indicative of the influx of HCO3− in exchange for Cl−. LEV (50 preincubation 15 min, n=5) had no effect on this type of alkalinization, whereas DIDS (500 μM, pretreatment 15 min, n=3) completely abolished it. LEV at a concentration of 50 μM does not inhibit the Na+-independent Cl−/HCO3− exchange.

LEV, at clinically relevant concentrations, induces the acidification of hippocampal neurons of adult guinea-pigs, an action most likely to be due to an inhibition of the Na+-dependent Cl−/HCO3− exchange. Electrophysiological experiments show that LEV decreases the frequency of action potentials and epileptiform bursting of CA3 neurons. This effect is switched off in the presence of LEV by a compensating intracellular alkalosis due to TMA treatment. Based on pHi studies on epileptic model systems, the anticonvulsive effect of LEV is at least in part attributable to an inhibition of acid extrusion.

This suggests that LEV inhibits Na+-dependent HCO3− transport in neurons. Evidence is based on the fact that both pHi regulation and steady-state pHi are lowered by LEV in the presence of an inwardly directed gradient of HCO3− and Na+, whereas pHi remains unchanged when these ions are absent from the extracellular fluid. Notably, LEV leaves the DIDS-sensitive Na+-independent Cl−/HCO₃− transport unchanged. Given that classical inhibitors of Cl−/HCO3− exchange such as DIDS are unable to distinguish between different types of Cl−/HCO3− exchangers, LEV likely serves as a selective inhibitor of Na+-dependent Cl−/HCO3− exchange. LEV may also influence Na+/HCO3− cotransport thought perhaps to be important for glial cells. But the amount of this exchanger within the somata of CA3 neurons is low, and its contribution to the maintenance of steady-state pHi and pHi recovery following acid load may be the subject of further research.

The effective concentration of LEV found in studies (10-50 μM) has been relatively low. Previously published effects of LEV on K+ channels were achieved with higher concentrations (100-500 μM). In spite of such high concentration, inhibition of K+ channels was limited to less than 20% (Madeja et al., 2003). Also, high-voltage-activated Ca2+ channels were moderately but irreversibly inhibited (−18%) by 100 μM LEV. Among these, N-type Ca2+ channel currents were the most sensitive and reduced by ca. 15% (calculated for 50 μM LEV). These minor changes may correspond to the obvious lack of changes observed for 4-AP-induced epileptiform potentials. But some channels are inhibited by intracellular acidosis. Among these are hippocampal KCNQ2/3 potassium channels and N-type calcium channels. Thus, effects on distinct currents may be secondary to low pHi. This could also explain why alkalosis following TMA restores original bioelectric patterns and counteracts effects of LEV.

LEV acidifies hippocampal neurons due to an impaired Na+-dependent Cl−/HCO3− exchange. As this intracellular acidification is sufficient to inhibit spontaneous epileptiform activity, the decrease of pHi by LEV likely contributes to its anticonvulsive property.

In some embodiments, the subject technology provides a pharmaceutical formulation or method, for treating cancer in a mammal, having active ingredients comprising at least two of: (a) a monocarboxylate transport inhibitor; (b) a sodium-hydrogen exchange inhibitor; (c) a chloride-bicarbonate exchange inhibitor; (d) a carbonic anhydrase inhibitor; or (e) a proton pump inhibitor; wherein those of (a) through (e) that are in the formulation are in amounts effective in combination to induce selective cytotoxicity in cancer cells relative to noncancerous cells in members of the same species as the mammal. In related embodiments, the pharmaceutical formulation further includes, or the method is further administered in combination with, one or more of the following drugs, compounds or dietary regimens.

Copper Chelators

A copper chelator may be an agent capable of creating a copper deficient environment, e.g., around a cancer cell or a tumor. A copper deficient environment may increase levels of surface Ctrl, resulting in increased cellular cisplatin uptake and reduced proliferation of a cancer cell.

Mutations in copper transporters such as in Wilson disease (export pump ATP7B) result in copper accumulation in the tissues and copper toxicity in several major organ systems. Copper chelation is necessary in patients with these diseases to reduce copper levels and toxicity. Accordingly, several copper chelators are approved for use in these patients, and may be used in the methods described herein to reduce copper levels.

Embodiments of the methods described herein provide for a copper chelator that binds copper in the Cu(I) or Cu(II) oxidation state. Some embodiments provide for a copper chelator having a higher binding affinity for Cu(I) relative to Cu(II). Some embodiments provide for a copper chelator having a higher binding affinity for Cu(II) relative to Cu(I). Copper chelators may include without limitation: penicillamine (Cuprimine®, Depen®), trientine hydrochloride (also known as triethylenetetramine hydrochloride, or Syprine®), dimercaprol, diethyldithiocarbamate (e.g., sodium diethyldithiocarbamate), bathocuproine sulfonate, and tetrathiomolybdate (e.g., ammonium tetrathiomolybdate). Suitably, a copper chelator may not have appreciable binding affinity for a platinum-based chemotherapeutic agent.

Tetrathiomolybdate, such as ammonium tetrathiomolybdate, may serve to chelate copper and may also compete with copper for intestinal absorption. Other agents used to control copper levels in patients with Wilson disease include zinc salts, such as zinc acetate

(Galzin®), which also compete with copper for intestinal absorption. Zinc may also induce production of metallothionein, a protein that binds copper and prevents its transfer into the bloodstream. Accordingly, tetrathiomolybdate and/or zinc may also be used to reduce copper absorption in the methods described herein.

Platinum-Based Chemotherapeutics

Platinum-based chemotherapeutic agents have been described as “the most important group of agents now in use for cancer treatment,” and are typified by cisplatin (cis-diamminedichloroplatinum (II)) (Reed, 1993, in Cancer, Principles and Practice of Oncology, pp. 390-4001), These agents, used alone or as a part of combination chemotherapy regimens, have been shown to be curative for testicular and ovarian cancers and beneficial for the treatment of lung, bladder, and head and neck cancers, among many others.

DNA damage is believed to be the major determinant of cisplatin cytotoxicity, though this drug also may induce other types of cellular damage. In addition to cisplatin, this group of drugs includes carboplatin and oxaliplatin, which like cisplatin are used clinically, and other platinum-containing drugs that are under development. These compounds are believed to act by the same or very similar mechanisms, so that conclusions drawn from the study of the bases of cisplatin sensitivity and resistance are expected to be valid for other platinum-containing drugs. Cisplatin is known to form adducts with DNA and to induce interstrand crosslinks. Adduct formation, through an as yet unknown signaling mechanism, is believed to activate some presently unknown cellular enzymes involved in programmed cell death (apoptosis), the process which is believed to be ultimately responsible for cisplatin cytotoxicity (see Eastman, 1990, Cancer Cells 2: 275-2802).

Embodiments of the methods described herein provide platinum coordination complexes wherein platinum is in the Pt(II) oxidation state. Some embodiments provide platinum coordination complexes having a square planar geometry with respect to the platinum atom.

Some platinum-based chemotherapeutics may include without limitation: cisplatin, carboplatin, oxaliplatin, iproplatin, tetraplatin, lobaplatin, dicycloplatin (DCP), PLD-147, JM118, JM216, JM335, and satraplatin. Such platinum-based chemotherapeutic agents also include the platinum complexes disclosed in EP 0147926, U.S. Pat. No. 5,072,011, U.S. Pat. Nos. 5,244,919, 5,519,155, 6,503,943 (LA-12/PLD-147), 6350737, and WO 01/064696 (DCP).

Autophagy Modulators

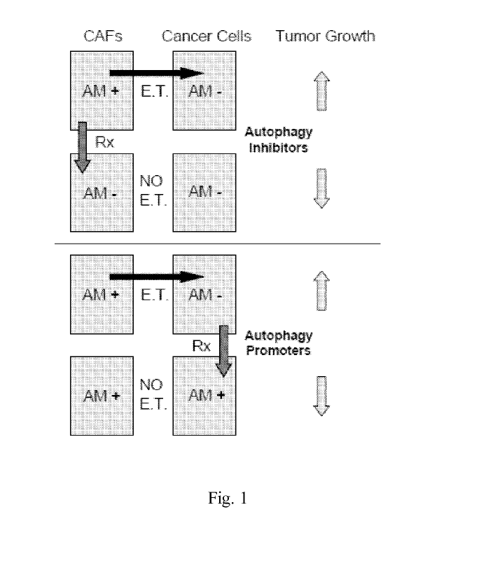

As used herein, the term “modulator” in relation to autophagy refers to any compound that can increase or decrease the rate of autophagy in a cancer, which can lead to the treatment, stopping, or slowing of the cancer's progression. Autophagy/mitophagy (AM) in the tumor stroma may sustain tumor growth. As shown in FIG. 1, the large black arrow signifies energy transfer (E.T.) from the stromal cancer associated fibroblasts (CAFs) to the epithelial cancer cells, via stromal autophagy/mitophagy. Thus, inhibition of autophagy in the tumor stroma would be expected to halt or reverse tumor growth. This could explain the effectiveness of known autophagy inhibitors as anti-tumor agents, such as chloroquine, hydroxychloroquine (Plaquenil®) and 3-methyladenine (Upper panel in FIG. 1). Conversely, induction of autophagy in epithelial cancer cells would also be expected to block or inhibit tumor growth (Lower panel in FIG. 1). This idea would also explain the anti-tumor activity of agents that activate autophagy, such as mTOR inhibitors, e.g. rapamycin. Thus, using this model, compounds that either systemically block or activate autophagy could both have the same net effect, which is to disrupt the metabolic coupling between the epithelial cancer cells and the tumor stromal fibroblasts. This model directly resolves the long-lived “autophagy paradox”, that both systemic inhibition of autophagy and systemic stimulation of autophagy have the same net effect, which is to inhibit tumor growth. See WO2012024612, where it is explained that autophagy, mitophagy, and aerobic glycolysis are all induced by oxidative stress and are all controlled by the same key transcription factor, namely hypoxia-inducible factor-1-alpha (HIF1-alpha). In this regard, the authors in (Liu et al., miR-31 ablates expression of the HIF regulatory factor FIH to activate the HIF pathway in head and neck carcinoma; Cancer Res 2010; 70: 1635-44.) have shown that a loss of stromal Cav-1 leads to the up-regulation of miR-31, which is a known activator of HIF1 alpha transcriptional activity. Thus, the lethality of a Cav-1-deficient tumor microenvironment could be explained by an autophagic/catabolic tumor stroma, which would then provide both nutrients and energy to epithelial cancer cells in a paracrine fashion. This is the “autophagic tumor stroma model of cancer”. This represents a unique therapeutic opportunity, as blocking autophagy in the tumor stroma should halt cancer growth, while an induction of autophagy in the epithelial cancer cells should have the same effect, thereby halting tumor growth. This new model of compartmentalized autophagy clarifies and explains the controversial role of autophagy in tumor pathogenesis. Other exemplary modulators of autophagy are disclosed in WO2011146879.

Glutaminolysis Inhibitors

Mammalian cells fuel their growth and proliferation through the catabolism of two main substrates: glucose and glutamine. Most of the remaining metabolites taken up by proliferating cells are not catabolized, but instead are utilized as building blocks during anabolic macromolecular synthesis.

Many cancer cell lines depend on a high rate of glucose uptake and metabolism to maintain their viability despite being maintained in an oxygen-replete environment. This metabolic phenotype has been termed aerobic glycolysis. Initially, this high rate of glycolysis was believed to result from mutations that impair the ability of cancer cells to carry out oxidative phosphorylation. However, such defects appear to be rare in spontaneously arising tumors. Recent studies have suggested that activating mutations in phosphoinositol 3-kinase (PI3K) and its downstream effector AKT induce the transformed cell to take up glucose in excess of its bioenergetic needs. The resulting high rate of glycolytic metabolism leads to the conversion of mitochondria into synthetic organelles that support glucose-dependent lipid synthesis and non-essential amino acid production. Glycolytic pyruvate that accumulates in excess of a cell's bioenergetic and synthetic needs is converted to lactate and secreted. A consequence of this metabolic conversion is that cells become addicted to glucose for their ATP production and survival as available lipids and amino acids are redirected from use as bioenergetic substrates and committed to use in anabolic synthesis.

In addition to glucose, glutamine can be an essential nutrient for cell growth and viability. In vitro addiction to glutamine as a bioenergetic substrate was first observed in HeLa cells, but it was not found to be a universal property of cancer cell lines. In cancer patients, some tumors have been reported to consume such an abundance of glutamine that they depress plasma glutamine levels. Despite these observations, the high rate of glutamine metabolism and addiction exhibited by some cancer cells is poorly understood. In such cells, the excess glutamine metabolites produced were found to be secreted as either lactate or alanine. This high rate of glutaminolysis was found to be beneficial because it provided the cell a high rate of NADPH production that was utilized to fuel lipid and nucleotide biosynthesis. However, not all tumor cells exhibit glutaminolysis. This suggested that the use of glutamine as a bioenergetic substrate is not induced as an indirect consequence of cell growth, but as a direct consequence of a specific oncogenic event.

Thus, glutaminolysis is a critical pathway for a host of cancers, especially those with Myc overexpression. Exemplary compounds that can inhibit this pathway (i.e., glutaminolytic inhibitors) include amino-oxyacetate (AOA), sodium phenylbutyrate, phenylbutyrate, phenylacetate, and 3,7-bis(dimethylamino)-phenazathionium chloride (methylene blue). See U.S. Publication No. 20110301153.

Glycolysis Inhibitors

As discussed above, glycolysis is an important pathways for cancer cells to maintain their viability. Therefore, glycolytic inhibitors can be effective in treating cancer. Exemplary glycolytic inhibitors include 2-deoxy-D-glucose (2DG), oxamate and various analogs thereof which are disclosed in U.S. Pat. No. 6,670,330, or those disclosed in U.S. Pub. No. 20100144652.

Somatostatin Receptor Binding Agents

Programmed cell death, so-called apoptosis, is an important instrument of the organism to prevent or combat cancer. Cells that have suffered irreparable damage to their DNA express the tumour suppressor protein p53, which induces cell apoptosis. About 50% of all human cancers are characterised by a mutation of p53 which saves the tumour cells from apoptosis.

Somatostatin is a cyclic peptide hormone which holds a key position in several regulatory metabolic processes. At present, five somatostatin receptors, SSTR1 to SSTR5, are known which may be allocated to the class of G-protein coupled receptors. By binding to these receptors, somatostatin, among other things, influences the adenyl cyclase activity, tyrosine phosphatase activity, MAP kinase activity, the regulation of K+ channels, Ca2+ channels and the activity of different phospholipases.

Somatostatin receptors, especially SSTR1-SSTR3, were also found on various tumour cell lines. For example, tumour cell lines of the pituitary gland (AtT-20), breast cancer cell lines (MCF7) and Langerhans tumour cell lines (Rin m5f, HIT) may be mentioned. Most human tumours also bear somatostatin receptors, usually in several isoforms.

Somatostatin has a very short half-life of just a few minutes in the human body so that it is hardly suitable as a therapeutic agent. Therefore, many efforts have been made to provide somatostatin derivatives that live longer in the human body. See, e.g., U.S. Pat. No. 5,480,879. There are indications already that somatostatin derivatives binding to somatostatin receptors may cause apoptosis of tumour cells. Therefore, influencing apoptosis with somatostatin derivatives is a promising approach for the therapy of cancer.

Several somatostatin derivatives are clinically applied in tumour therapy already. Examples of this derivatives are octreotide, vapreotide and seglitide. See, e.g., U.S. Pat. No. 5,480,870. Other exemplary somatostatin receptor binding agents, such as cyclic or linear tetra- or pentapeptides, are disclosed in U.S. Pub. No. 20030114362.

Ketogenic Diet

The human body derives the energy that is needed predominantly from eating and metabolizing proteins, lipids and digestible carbohydrates. In the typical Western diet, digestible carbohydrates provide most of the energy, in most cases more than 50 energy percent, using 4 kcal or 16.8 kJ per gram as the conversion factor for digestible carbohydrates and proteins and 9 kcal or 37.8 kJ per gram for lipids. Metabolism of digestible carbohydrates predominantly releases glucose, which is a preferred energy source for the human body. Under special conditions most human cells can also use other organic compounds as energy source, such as amino acids, fatty acids and ketones. It is generally considered that increasing the amount of carbohydrates relative to that of lipids decreases the ketogenic properties of the product. Therefore classical ketogenic products for paediatric epileptics provide 4 times more lipids than the sum of proteins and digestible carbohydrates calculated on a weight base.

A ketogenic diet is a diet that induces an organism to use ketones as a major energy source. These ketonic compounds include acetoacetate, D-3 hydroxybutyrate, and acetone. Ketoacids like oxaloacetate are not calculated as ketobodies. In particular a ketogenic diet comprises more than 60 g lipids per 100 g dry mass of the dietetic products, so more than 77 percent of energy.

Ketogenic diets have been used for treatment of epileptic convulsions and in treatments of obesity and weight management. Such diets may be composed of a variety of different meals which each fit within the diet, or may consist of one single product, which can be used for complete nourishment of a human being, when used as the sole nutrition. A product called Ketocal belongs to the latter category and is used for combating epilepsy, in particular intractable epilepsy in young infants. The product provides per 100 g dry matter 3011 kilojoules and comprises per 100 g dm 15.25 g protein, 73 g lipids and 3 g digestible carbohydrates. The amount of saturated fatty acids is about 22 wt % of the triglycerides. However, the low amount of carbohydrates and the nature of the lipid made the product suboptimal for human consumption. It is therefore an object of the subject technology to provide a highly effective product that is more palatable, while providing a better lipid fraction in terms of ketogenic properties, tolerance, safety and a lower amount of trans fatty acids.

EP 0843972 discloses a product for enteral feeding of persons suffering from metabolic syndrome or hypertriglyceridemia, which comprises 33-63 energy percent fats, and the proteins 5-30 energy % of the composition, and in which the fatty acids comprise 55-90 wt % medium chain fatty acids, 5-25 wt % polyunsaturated fatty acids, and 0-30 wt % other fatty acids. The levels of palmitic acid are low, on the order of 0.5-1% on fatty acid basis. Exemplary ketogenic diets are disclosed in U.S. Pub. Nos. 20110301238, 20100310740, 20080089981 or EP 2073467 B1.

pH-Dependent Drug Delivery

pH-sensitive polymeric micelles and nanogels can target a slightly acidic extracellular pH environment of solid tumors. The pH targeting approach is regarded as a more general strategy than conventional specific tumor cell surface targeting approaches, because the acidic tumor microclimate is most common in solid tumors. When nanosystems are combined with triggered release mechanisms by endosomal or lysosomal acidity plus endosomolytic capability, the nanocarriers can overcome multidrug resistance of various tumors. As used herein, a “micelle” can refer to its ordinary meaning and, in some cases, can refer to nanoparticles generally.

The extracellular pH (pHe) of normal tissues and blood pH are kept relatively constant at about pH 7.4 and their intracellular pH (pHi) at about 7.2. In most tumors the pH gradient is reversed (pHi>pHe). Particularly, tumor pHe is lower than normal tissues. Although there is a distribution in vivo, pHe measurements made by using needle-type microelectrodes on human patients having various solid tumors (adenocarcinoma, squamous cell carcinoma, soft tissue sarcoma, and malignant melanoma) and in readily accessible areas (limbs, neck, or chest wall), shows the mean pHe value to be 7.0 with a range between 5.7 and 7.8.

This variation is dependent upon tumor histology, tumor volume, and location inside a tumor. Measurements of pHe by noninvasive technology such as 19F, 31 P, or 1H probes by magnetic resonance spectroscopy in human tumor xenografts and in animals further proved consistently low pHe. Reported pHe data on human and animal solid tumors either by invasive or noninvasive methods showed that more than 80% of all measured values are below pH 7.2. The primary reason for this imbalance in cancer pH is the high rate of glycolysis in cancer cells, both in aerobic and anaerobic conditions. The acidic milieu benefits cancer cells by generating an invasive environment that tears down the extracellular matrix and destroys the surrounding normal tissue cells.

A variety of mechanisms associated with multidrug resistance (MDR) cells need to be circumvented for a successful tumor treatment. At a unicellular level, ATP dependent drug-efflux pumps of P-glycoprotein (Pgp), multidrug resistance protein (MRP), lung resistance protein (LRP), antiapototic (or survival) bcl-2 gene, and altered expression of Topoisomerase II interfere with a sufficient intracellular drug dose and decrease the effectiveness of drugs in killing tumor cells. In the clinical setting, additional tumor microenvironmental factors such as epidermal growth factor, fibroblast growth factor, insulin-like growth factor, and extracellular matrix components are strongly associated with survival mechanisms of cancer cells under cytotoxic drug treatment.

Multifaceted MDR mechanisms may require a focal high dose strategy that works at cellular level rather than at systemic level. Local high doses may overwhelm most resistant mechanisms, which might have an intrinsic limitation in defense capability because even extremely resistant experimental MDR cells are killed at high drug concentrations. Intracellular organelles in parental drug-sensitive cells are characterized to have somewhat acidic, diffuse pH profiles inside cells. MDR cancer cells develop more acidic organelles (recycling endosome and lysosome) than those in sensitive cells, which are more acidic than cytosolic pH and nucleoplasmic pH. This results in acid-induced sequestration of anticancer drugs. Acidic organelles in MDR cells contribute to developing resistance to chemotherapeutic drugs. Because most anticancer drugs are in an ionizable form, the pH of extracellular matrix and intracellular compartments are critical factors in determining drug partitioning and distribution. The low pH in tumor extracellular space or in various subcellular organelles is a significant signal for targeting.

Extracellular pH (pHe) Targeting

The approaches to target various solid tumors by pHe include micelle systems with a triggered drug release mechanism, and exposing nonspecific cationic TAT (HIV transactivator of transcription) peptide by a shielding/deshielding mechanism or by a pop-up mechanism. These

Systems have utilized the pH-sensitivity of poly(L-histidine) or polysulfonamide. The imidazole ring of a polyHis (pKb˜7.0; polyHis is the most effective pH-buffering agent in a physiological system) has lone pairs of electrons on the unsaturated nitrogen that endow pH-dependent amphoteric properties. Particular polysulfonamides (pKa˜6.8) are negatively charged at blood pH (i.e., pH 7.4) and can be neutralized at acidic pH (e.g., tumor pHe). In addition, these polymers demonstrated a strong endosomolytic property by proton sponge effect and/or interactions with the anionic phospholipids of endosome. These properties of polyHis or polysulfonamide enable nanosystems designed for targeting tumor pH.

Triggered pHe Drug Release

pH-induced anticancer drugs can be released from pH-sensitive liposomes, which are stable at neutral pH but leaky under mild acidic condition (pH 4.5-6.0), and can serve as a modality for tumor treatment. However, due to the lack of response to tumor acidity (pH 6.5-7.2), these carriers may not be optimal for pHe targeting. Polymeric micelles that have the capability of responding to tumor pHe have been designed. These polymeric micelles are physically destabilized and thus accelerate the anticancer drug release at tumor pHe.

A mixed pH-sensitive micelle (PHSM) system with folate (PHSM/f), created from poly(L-histidine) (polyHis) (Mw 5000)-b-poly(ethylene glycol) (PEG) (Mw 2000) and poly(L-lactic acid) (PLLA) (Mw 3000)-b-PEG (Mw 2000)-folate (0-25 wt. %), shows a gradual destabilization below pH7.0 due to the ionization of the polyHis block in the micelle core. In drug release studies, PHSM/f containing 25 wt. % PLLA-b-PEG shows a favorable pH-dependency, such that within 24 h, 32 wt. % of doxorubicin (DOX) is released at pH 7.0, 70 wt. % of DOX at pH 6.8, and 82 wt. % at pH 5.0. This enhances the killing effect on sensitive cancer cells below pH7.0. Furthermore, the DOX-loaded PHSM/f (equivalent DOX=10 mg/kg) exhibits significant inhibition (Pb0.05 compared with free DOX or saline solution) on the growth of s.c. MCF-7 xenografts. The tumor volume of mice treated with the PHSM/f (Pb0.05 compared with free DOX) is approximately 4.5 to 3.6 times smaller than those treated with saline solution or free DOX after 6 weeks.

Using DOX-loaded polyHis(Mw 5000)-co-PEG (Mw 3000) micelle, without folate and mixing with PLLA-b-PEG, in MDA 231 MD breast tumor-bearing mice model, the time-dependent DOX accumulation may be visualized using a skinfold window chamber model. The intensity of DOX fluorescence carried by PHSM is significantly more intense and spread within the tumor site than that carried by a control pH-insensitive (PLLA-b-PEG) micelle. This is because once the micelles are exposed to pHe, the micelles dissociate and release their payload. The micelle dissociation may also help the extravasation of next-arriving micelles by providing space. Therefore, the pH-induced micelle destabilization and triggered release of DOX by tumor pHe, after the accumulation of the micelles in the tumor sites via enhanced permeability, presents a more effective modality of chemotherapy for sensitive tumors by providing higher local concentrations of the drug at tumor sites and minimal release of the drug from micelles during blood circulation (pH 7.4).

The detailed physicochemical characteristics of a mixed PHSM, including the size, pH-dependent size change-dissociation kinetics, stability, the compatibility of core forming polymer blocks are established, as are PK data using polyHis-b-PEG micelles.

Shield/Deshielding Mechanism

The shield/deshielding mechanism by positive charges of TAT, a cell penetrating peptide, on micelle surfaces controlled by the pH difference between 7.4 and pHe has been designed. Poly(methacryloyl sulfadimethoxine)-b-PEG is negatively charged and interacts electrostatically with TAT molecules (shielding) at pH 7.4. However, charge density on this polymer decreases by decreasing pH. Below pH 6.8, due to destabilized electrostatic interactions, the TAT is deshielded. The zeta potential measurements on micelle comprising of PLLA-b-PEG-TAT demonstrates the shield/deshielding process. The zeta potential is close to zero between pH 8.0 to 6.8, which indicates complete shielding of TAT, and from pH 6.6 to 6.0 it increases to 6.0 mV, which is close to the measured zeta potential for TAT decorated micelles without masking. When the shielded and unshielded TAT micelles are tested for tumor cell internalization at pHs 7.4 and 6.6 by incubating for an hour, unshielded micelles are internalized into both the cells and its nucleus at pH 7.4 and 6.6. However, the micelle shielded with poly (methacryloyl sulfadimethoxine)-b-PEG is not internalized at 7.4, indicating TAT is masked even as it internalized into cells and the nucleus at pH 6.6. This shield/deshielding mechanism suggests that an optimized pHe targeting system with an appropriate sulfonamide polymer can be tailored for particular clinical use.

Endosomal pH (pHendo) Targeting

Accelerated anticancer drug release from L-histidine-based polymeric micelles could be triggered by an early endosomal pH of 6.0. The primary objective of this strategy is to create drug-loaded micelles that destabilize at an early endosomal pH of 6.0, such that drug release at both the tumor extracellular pH (pHe) and the lysosomal pH of 5.0 can be minimized. This system is effective for cytosolic high dose drug delivery with minimal drug loss during circulation and in the extracellular domain. The endosolytic activity at early endosomal pH may minimize leakage of digestive lysosomal enzymes.

Receptor-Mediated Endocytosis and pHendo Targeting

When a mixed PHSM/f micelle with up to 40 wt. % PLLA-b-PEG is used, a fraction of this micelle, depending on PLLA-b-PEG content, is destabilized by tumor extracellular pH, and the remaining is internalized by folate receptor-mediated endocytosis. To eliminate this micelle destabilization before internalization, a micelle system consisting of poly (His-co-phenylalanine (Phe))-b-PEG and PLLA-b-PEG-folate can be utilized. The pH-sensitivity of the micelle is controlled by the His/Phe block composition, and is fine tuned to target early endosomal pH through blending with PLLA-b-PEG by using anticancer drug, DOX.

Because pKb of poly(His-co-Phe (16 mol %))-b-PEG is around 6.3, this block copolymer can be blended with 20 wt. % of PLLA-b-PEG-folate for targeting endosomal pH of 6.0. This micelle (as denoted ‘EndoPHSM/f’) releases minimal drug above pH 6.0 and demonstrates the triggered release at pH 6.0, indicating tuned micelle destabilization at early endosomal pH. When

EndoPHSM/f was internalized into tumor cells via folate receptor-mediated endocytosis, this system effectively kills tumor cells through a focal high dose of DOX in the cytosol, resulting from active internalization, accelerated DOX release triggered by endosomal pH, and disruption of endosomal membrane.

An exemplary carrier system consists of two components, poly(L-lactic acid)-b-PEG-TAT micelles and pH-sensitive poly(methacryloyl sulfadimethoxine)-b-PEG. At normal blood pH, polysulfonamide is negatively charged, and when mixed with TAT, polysulfonamide shields the TAT by electrostatic interaction. Only PEG is exposed to the outside which could make the carrier long circulating. When the system experiences a decrease in pH (near tumor) polysulfonamide loses charge and detaches, exposing TAT for interaction with tumor cells.

Premature drug release in blood leads to systemic side effects and fails to concentrate at the site of action. Especially for drug-resistant cells, slow drug release kinetics in tumors may decrease the drug efficacy at the site. Consequently, a drug delivery system is needed for the intracellular focal high dose targeting based on triggered release mechanism to achieve maximal therapeutic efficacy.

After identification of Pgp as a major cause of MDR in in vitro cell studies and the discovery that verapamil modulates Pgp, efforts have been made for the identification of more effective Pgp modulators, which have high binding affinity to Pgp without disruption to normal biological functions and with minimal pharmacokinetic interference. Numerous Pgp modulators have been synthesized by chemical modification using existing drugs such as verapamil, cyclosporine A, glibenclamide, and other compounds. As an alternative to Pgp modulators, various drug carriers have been tested to overcome MDR in vitro and in vivo.

EndoPHSM/f micelles are highly effective in treatment of MDR tumor cells. The endosomal pH triggering anticancer drug release and the endosomal escaping activity of polyHis allows cytosolic delivery of anticancer drug, by avoiding drug sequestration mechanism in MDR cells and bypassing MDR protein expression on cellular membranes via folate receptor-mediated endocytosis. The EndoPHSM/f demonstrates a similar degree of cytotoxicity against MDR tumor cells (MCF-7/DOXR with Pgp overexpression) when compared with free DOX against the drug-sensitive tumor cells. Investigation of A2780/AD (ovarian carcinoma drug-resistant tumor) xenografts in nude mice for in vivo efficacy demonstrates the tumor regression in mice treated by EndoPHSM/f is promising and superior to PHSM/f.

Targeting both pHe and pHendo

To address MDR and tumor heterogeneity, one can use tumor cell nonspecific interactions that can be activated by the tumor microclimate, such as pH (pHe targeting) and with triggered release in endosomes.

Polymeric micelles with pH-induced ligand repositioning on the micelle surface can be used. The mixed micelle consists of polyHis-b-PEG and PLLA-b-PEG-b-polyHis-biotin, which is multifunctional; the shorter polyHis block in PLLA-b-PEG-b-polyHis-biotin is located at the interface of the hydrophobic core of PLLA and polyHis and the hydrophilic PEG shell, due to the high water solubility of neighboring PEG and biotin. The interfacial polyHis causes PEG chain bending and biotin burying in the PEG shell, derived from the polyHis-b-PEG block copolymer. As a result, the micelle is stable above pH 7.2 and hides the conjugated biotins. However, as the pH is lowered below pH 7.2, the degree of ionization of polyHis increases.

The interfacial short polyHis (Mw 1000) becomes ionized first, and at the critical degree of ionization its hydrophobic interaction with the core phase weakens. As a result, the PEG-b-polyHis-biotin portion expands, exposing biotin out of the PEG shell. The pH 7.0 appears to be the point for this expansion, as demonstrated by pH-dependent turbidity of the micelle solution containing avidin, which is a tetrameric protein with four biotin-binding sites. Furthermore, when the solution pH is decreased, the relative transparency of the solution is gradually reduced to 10% between the pH range of 6.8-6.0. This is attributed to ionization of the polyHis block located in the core and subsequent micelle destabilization by ionized polyHis escaping from the micelle.

This process might reduce transparency, presumably through a degree of aggregation of the remaining PLLA-b-PEG block copolymer. In summary, when the environmental pH for the micelle is lowered slightly (pH˜7.0; tumor acidic pH), biotin is exposed on the micellar surface and can interact with cells, which facilitates biotin receptor-mediated endocytosis. When the pH is lowered further (pH<6.5), the micelle destabilizes, resulting in disruption of the endosomal membrane and enhanced cytosolic drug release. The hidden biotin at pH 7.4 is exposed at pH 7.0 by the pop-up mechanism of the micelle, enhancing the cell cytotoxicity of the DOX-loaded micelle at tumor acidic pH.

While above pH 7.0, biotin anchored on the micelle core via a pH-sensitive molecular chain actuator (polyHis) is shielded by PEG shell of the micelle; biotin is exposed on the micelle surface (6.5<pH<7.0) and can interact with cells, which facilitates biotin receptor-mediated endocytosis. When the pH is further lowered (pH<6.5), the micelle destabilizes, resulting in enhanced drug release and disrupting cell membranes such as endosomal membrane.

To replace biotin with TAT, a micelle system can be constructed with polyHis-b-PEG and PLLA-b-PEG-b-polyHis (Mw 2000)-TAT. TAT is a non-specific cell penetrating peptide, which has the capability to translocate polymeric micelles into cells.

pH-dependent micelle uptake by cells occurs. At pH 7.4, micelle uptake is minimized. At pH 7.0, the uptake shows a 30-fold increase compared to pH 7.4, which probably is due to partial TAT expression on micellar surface. At pH 6.8, 70-fold increased micelle cellular uptake was shown as compared to pH 7.4. At tumor pHe, the TAT peptide is exposed on the micellar surface and interacts with cells, which facilitates macropinocytosis. This nanosystem is effective for various in vivo solid tumors including drug-sensitive and drug-resistant phenotypes and can replace selective antibody or ligand-based targeting technology.

Virus-Mimetic Nanosystems

Viruses infect specific cells of host organisms, replicate, destroy the cells, and spread from one to another cell, causing disease. They circulate long in the blood and, over time, become more pathogenic. Drug delivery vehicles often mimic only a few aspects of viruses, such as size and surface modifications for longer residence in the body before their clearance. Nanosystems having virus-like infectious properties, such as virus-mimetic (VM) nanogels, can be illustrated. Such a system has a capsid-like protein capsule, able to infect specific cells, injects toxin, destroys infected cells, and migrates to neighboring pathologic cells by repeated cell cycles. An exemplary virus-like infectious nanogel consists of a hydrophobic core (poly (His-co-Phe)) and two layers of hydrophilic shells (PEG and bovine serum albumin (BSA)). One end of PEG is linked to the core forming block and other to BSA, which forms a capsid-like outer shell. The structure of core and inner shell is formed by an oil-in-water emulsion method. At high pH, the core of this nanogel is rigid; however, the core swells by the ionization of polyHis at low pH. When these nanogels are exposed to early endosomal pH of 6.4, the size grows abruptly, reaching about 360 nm.

The size changes by cycling pH between 7.4 and 6.4 are also reversible. This reversible swelling/deswelling by pH of the core is closely linked to the release rate of incorporated DOX. The nanogels release a significant amount of DOX at endosomal pH (e.g., pH 6.4), while reducing DOX release rates at cytosolic or extracellular pH (e.g., pH 7.4-6.8). Due to a proton buffering effect of polyHis and observed substantial nanogel volumetric expansion within cell endosomes, nanogels are proposed to be able to physically disrupt endosomal membranes. This allows the VM nanogels and anticancer drugs released from the nanogels to transfer from the endosomes to the cytosol, where the VM nanogels rapidly shrink back to their original size with a more neutral local pH, and thereby reduce the drug release rate. Free drug released by endosomal pH stimulus is in the cytosol, then diffuses into the nucleus, and finally to the pharmacological target site. The drug can induce, e.g., apoptosis and eventually disintegrate. This releases the nanogels from the cell for subsequent infection and action in neighboring cells. This nanogel demonstrates repeated infectious cycles in cultures of drug-resistant tumor cells.

This approach can maximize drug efficacy in treating tumors, inflammation, and other diseases due to sequential cytotoxic action. Tumor extracellular pH- and/or endosomal pH-responsive micelles, TAT shield/deshield nanosystem, virus-like infectious nanogels, and pop-up micelles are examples of anticancer drug delivery systems that overcome limitations of conventional drug delivery. These systems increase target drug accumulation at tumor sites or at intercellular cytosolic compartments in tumor cells, with less drug distribution to normal tissues and organs. In particular, the pH-sensitive micelles or nanogels described are effective delivery systems in the treatment of cancer, including MDR cells.

Controlled Release

Some aspects of the subject technology relate to controlled-release preparations. Some embodiments include a capsule comprising a tablet, granule, or fine granule, wherein the release of active ingredient is controlled by pH, time, or both pH and time. Some embodiments include a gel-forming polymer that delays migration speed in the gastrointestinal tract.

An oral formulation is a dosage form used frequently among pharmaceutical agents. Useful preparations for oral administration sustain drug efficacy with administration once or twice a day, aimed at improving quality of life. For oral controlled-release preparations, various release-control systems can be made, such as a release-controlled coating-layer or a diffusion control of compound by a matrix, a release control of compound by erosion of matrix (base material), and a pH-dependent release control of a compound and a time-dependent release control wherein the compound is released after a certain lag time. A further extension of sustainability becomes possible by combining any of the above-mentioned release-controlled systems with a control of migration speed in the gastrointestinal tract.

After administered orally, a tablet, granule, or fine granule can migrate through gastrointestinal tract, releasing an active ingredient to stomach, duodenum, jejunum, ileum, and colon sequentially. A controlled release preparation is designed to control the absorption by delaying the release of active ingredient in some way. It is considered that a further extension of sustainability becomes possible, by combining a release-controlled system with a function to control the migration speed in gastrointestinal tract such as adherability, floatability etc. Disclosure is contained in, e.g., WO 01/89483, U.S. Pat. No. 6,274,173, U.S. Pat. No. 6,093,734, U.S. Pat. No. 4,045,563, U.S. Pat. No. 4,686,230, U.S. Pat. No. 4,873,337, U.S. Pat. No. 4,965,269, U.S. Pat. No. 5,021,433, each of which is incorporated by reference herein it its entirety.

Aspects of the subject technology provide a controlled release preparation wherein release of active ingredient of drug is controlled, releasing an active ingredient for an extended period of time, staying or slowly migrating in the gatrointestinal tract. A capsule comprising a tablet, granule or fine granule wherein the release of active ingredient is controlled and a gel-forming polymer.

Capsules in some embodiments can include a gel-forming polymer whose viscosity, as a 5% aqueous, solution, is about 3,000 mPas or more at about 25 degrees C. Capsules in some embodiments can include a gel-forming polymer, such as one having a molecular weight of 400,000 to 10,000,000 daltons.

Capsules in some embodiments can include one of more layers having a release-controlled material, such as one or more kinds of polymeric substances such as hydroxypropylmethyl cellulose phthalate, cellulose acetate phthalate, carboxymethylethyl cellulose, methyl methacrylate-methacrylic acid copolymer, methacrylic acid-ethyl acrylate copolymer, ethyl acrylate-methyl methacrylate-trimethylammoniumethyl methacrylate chloride copolymer, methyl methacrylate-ethyl acrylate copolymer, methacrylic acid-methyl acrylate-methyl methacrylate copolymer, hydroxypropyl cellulose acetate succinate, and/or polyvinyl acetate phthalate.

In some embodiments, pH-dependently soluble release-controlled coating-layer can include, e.g., cone kind of polymeric substance or a mixture of two or more kinds of polymeric substances having different release properties such as hydroxypropylmethyl cellulose phthalate, cellulose acetate phthalate, carboxymethylethyl cellulose, methyl methacrylate-methacrylic acid copolymer, methacrylic acid-ethyl acrylate copolymer, methacrylic acid-methyl acrylate-methyl methacrylate copolymer, hydroxypropyl cellulose acetate succinate, polyvinyl acetate phthalate and shellac. The polymeric substance can be selectively soluble in various pH ranges, such as a pH range of 6.0 to 7.5.

In some embodiments, a substantially water soluble crystalline polymer of a matrix comprises a crystalline polyethylene glycol polymer having dispersed therein at least one non-ionic emulsifier as the surface active agent. A suitable matrix for use in some compositions of the subject technology is one of the type described in WO 89/09066 or WO 91/04015, to which reference is made and which are incorporated herein by reference, i.e. a matrix containing a crystalline polyethylene glycol polymer, typically with a molecular weight of at least about 20,000 daltons, in which at least one non-ionic emulsifier is dispersed. Suitable non-ionic emulsifiers include, e.g., fatty acid esters and/or fatty acid ethers, for example a fatty acid ester and/or fatty acid ether having carbon chains of from 12 to 24 carbon atoms, typically from 12 to 20 carbon atoms, such as an ester and/or ether of palmitic acid or stearic acid. Examples are polyglycol esters and ethers, polyethylene glycol esters and ethers, polyhydroxy esters and ethers, and sugar esters and ethers such as a sorbitan ester or ether.

A suitable HLB (hydrophilic-lipophilic balance) value is in the range of from about 4 to about 16. The non-ionic emulsifier is preferably approved for use in products to be ingested by humans or animals, i.e. pharmaceuticals and/or foodstuffs. A preferred non-ionic emulsifier for use in the matrix is polyethylene glycol stearate, in particular a polyethylene glycol monostearate such as polyethylene glycol 400 or 2000 monostearate. Tartaric acid, citric acid and lactic acid esters of mono- and diglycerides, as well as fatty acid esters of glycerol may also be employed. The matrix may in addition include a cellulose derivative, e.g. a cellulose derivative selected from the group consisting of methylcellulose, carboxymethylcellulose and salts thereof, microcrystalline cellulose, ethylhydroxyethylcellulose, ethylmethylcellulose, hydroxyethylcellulose, hydroxyethylmethylcellulose, hydroxypropylcellulose, hydroxypropylmethylcellulose, hydroxymethylcellulose and hydroxymethylpropylcellulose. Of these cellulose derivatives, hydroxypropylmethylcellulose and methylcellulose are preferred for incorporation in the matrix.

Although an amount of surface active agent will vary depending on such factors as the nature of the surface active agent and the desired dissolution characteristics of the matrix, the surface active agent will typically be present in an amount of about 1-40% by weight of the matrix, more typically about 2-30%, e.g. about 4-20%, such as about 5-15%.

Some crystalline polyethylene glycol polymers for use in the matrix have a molecular weight in the range of 20,000-35,000 daltons, although some compositions according to the subject technology will also include those in which the matrix contains a polyethylene glycol polymer with a molecular weight of less than 20,000 daltons, e.g. in the range of about 10,000-20,000 daltons.

The crystalline polymer matrix must have a melting point which is above the temperature of the aqueous medium in which a composition of the subject technology is to be used. Thus, for the delivery of a drug for human or veterinary use, the matrix can suitably have a melting point of about 40-80 degrees C.

Where reference is made herein to the fact that a release modifier functions to regulate erosion of a matrix within a pH range of from, e.g., about 2 to about 7, this means that the release modifier is one which, due to its pH-dependent solubility, provides the matrix with different degrees of erosion at different pH values within this range. Typically, the release modifier will be a compound that is soluble above a given pH in the range of from about 5 to about 7, e.g. a pH of about 5.0, 5.5, 6.0, 6.5 or 7.0, but substantially insoluble at lower pH values.

A release modifier may be selected from materials conventionally used in the pharmaceutical industry to produce enteric coatings. A number of different types of compounds suitable for use as enteric coatings are known in the art; see e.g. Remington's Pharmaceutical Sciences, 18.sup.th Edition, 1990. Release modifiers may in particular be selected from one of three general classes, namely cellulose derivatives, methacrylic acid polymers and modified gelatine compounds. Preferred release modifiers include cellulose acetate phthalate, polyvinyl acetate phthalate, hydroxypropyl methylcellulose phthalate and hydroxypropyl methylcellulose acetate succinate, as well as methacrylic acid copolymers. Modified gelatine compounds include gelatine treated with e.g. formaldehyde or glutaraldehyde. Examples of commercially available polymers suitable as release modifiers are EUDRAGIT® L and EUDRAGIT® S, available from Rohm GmbH, Germany, and enteric coating agents available from Shin-Etsu Chemical Co., Japan. The release modifier will typically be present in the composition in an amount of about 0.1-10%, based on the weight of the matrix, preferably about 0.5-4%, e.g. about 1-3%, such as about 1.5-2.0%. If desired, a suitable mixture of more than one release modifier may be used in order to obtain a desired release profile in any given composition.

In some embodiments, controlled release compositions of the subject technology further comprises a coating having at least one opening exposing at least one surface of the matrix, the coating being one which crumbles and/or erodes upon exposure to the aqueous medium at a rate which is equal to or slower than the rate at which the matrix erodes in the aqueous medium, allowing exposure of said surface of the matrix to the aqueous medium to be controlled. Coatings of this type are described in WO 95/22962, to which reference is made and which is incorporated herein by reference. These coatings can comprise: (a) a first cellulose derivative which has thermoplastic properties and which is substantially insoluble in the aqueous medium in which the composition is to be used, e.g. an ethylcellulose such as ethylcellulose having an ethoxyl content in the range of 44.5-52.5%, or cellulose acetate, cellulose propionate or cellulose nitrate; and at least one of (b) a second cellulose derivative which is soluble or dispersible in water, e.g. a cellulose derivative selected from the group consisting of methylcellulose, carboxymethylcellulose and salts thereof, cellulose acetate phthalate, microcrystalline cellulose, ethylhydroxyethylcellulose, ethylmethylcellulose, hydroxyethylcellulose, hydroxyethylmethylcellulose, hydroxypropylcellulose, hydroxypropylmethylcellulose, hydroxymethylcellulose and hydroxymethylpropylcellulose; (c) a plasticizer, e.g. selected from the group consisting of phosphate esters; phthalate esters; amides; mineral oils; fatty acids and esters thereof with polyethylene glycol, glycerin or sugars; fatty alcohols and ethers thereof with polyethylene glycol, glycerin or sugars; and vegetable oils; or a non-ionic surfactant; or (d) a filler, e.g. selected from conventional tablet or capsule excipients such as diluents, binders, lubricants and disintegrants.

A coating of this type may in addition further comprise a release modifier of the type described above, so that the coating is provided with an erosion profile similar to that of the matrix in terms of the relative rate of erosion in the stomach and the intestines, respectively. In this case, it may be advantageous to incorporate a somewhat higher concentration of the release modifier in the coating than the concentration of release modifier in the matrix, so as to ensure that the coating does not erode in the stomach at a faster rate than the matrix.

In some embodiments, compositions can be adapted to compensate for differential absorption in the gastrointestinal tract or to provide different rates of release of the active substance in the small intestine and in the large intestine, e.g. by varying the concentration of the release modifier or the active ingredient in different zones of the matrix. The exact release profile provided by any given matrix in a controlled release composition of the subject technology can be dependent on the nature of the matrix, including the type and amount of crystalline polymer, surface active agent and release modifier, as well as the nature and amount of the active ingredient in the matrix and the characteristics of a possible coating. However, by adjusting in particular the concentration of the release modifier and the active ingredient, and using routine testing of appropriate variations in vitro and in vivo, a person skilled in the art will readily be able to arrive at compositions that provide a desired release profile for a given active substance under a given set of circumstances.

For example, for obtaining a composition with a first release rate in the small intestine and a second release rate in the large intestine (the small intestine typically having a slightly higher pH value than the large intestine, i.e. normally about 7.2 and 6.9, respectively), a release modifier which is soluble at a pH of from about 7.0 or 7.1, but which is substantially insoluble or at least substantially less soluble at pH values below 7.0, may be chosen. In this case, the composition will comprise at least one first zone with a first concentration of the release modifier and optionally at least one second zone with a second concentration of the release modifier. An example of a suitable release modifier for this purpose is EUDRAGIT® S, available from Rohm GmbH, Germany.

In some cases, an active substance can be substantially homogeneously distributed throughout the matrix. A matrix of this type is simple to produce, and for many purposes a simple release profile obtained in this manner will be sufficient. In other cases, however, it will be desired to have at least two matrix zones having different concentrations of the active substance, since this makes it possible to provide more complex release profiles, for example a pulsatile or second burst release of the active substance. Many different variations on such a composition can of course be contemplated, e.g. a composition comprising, in addition to at least one matrix zone comprising an active substance, at least one remote zone comprising an active substance, optionally dispersed in a filler, such that the remote zone becomes exposed to the aqueous medium after a predetermined period of, e.g., at least about 15 minutes after administration of the composition. These types of more complex compositions may also include two or more different active substances in two or more different zones of the composition. Different release patterns (i.e. zero order and pulsatile) may also be combined so that a uniform release of one active substance (for example at a fairly low dosage level) alternates with the release in bursts of the same or another active substance (for example at a higher dosage level). Other variations will be apparent to persons skilled in the art.

Due to the nature of the matrix in some embodiments, diffusion of water into the composition of the subject technology is substantially limited to any exposed surface layers of the matrix, and release of the active substance is therefore proportional to the rate of erosion of such exposed matrix surfaces. Since the subject technology makes it possible to adapt the erosion rate taking into consideration the different pH, agitation, absorption and residence time conditions existing in the stomach and intestines, the release profile of any given composition can similarly be adapted as necessary. In particular, the subject technology makes it possible to obtain an approximately zero order release profile over an extended period of time, e.g. up to about 24 hours or even longer, in spite of the significantly different conditions to which a composition is exposed during such an extended time period while passing through the various portions of the gastrointestinal system. This is an advantage that improves the utility and range of possible uses for such controlled release compositions.

Cancer Patient Case Reports using pH-Manipulation Therapy

Patients have been treated orally with a combination of drugs that inhibit the efflux of acid (H+, CO2, and/or lactic acid). Although these drugs tend to alkalinize the extracellular milieu of cancer cells, they may be administered in combinations that achieve a metabolic acidosis.

Esomeprazole (vacuolar H+ ATPase pump inhibitor) was used either daily or every other day, with a dose ranging from 240 mg per day up to 320 mg per day, or more.

Methazolamide (a carbonic anhydrase inhibitor) was used every other day with a dose ranging from 50 mg per day up to a maximum of 400 mg per day. Breaks were taken from methazolamide therapy to decrease the likelihood of, or to ameliorate, side effects such as tingling in extremities, acidosis, fatigue, and nausea. Side effects were especially noticeable in a patient with elevated liver function tests due to metastatic disease to the liver.

Amiloride (Na−H+ exchanger inhibitor and an inhibitor of urokinase plasminogen activator) was used daily at a dose of 20 mg per day, or more. Because amiloride is a potassium-sparing diuretic, with a propensity to cause hyperkalemia, HCTZ, at 25 mg 2 times per day was administered as well. In addition, the use of amiloride in combination with HCTZ has shown to inhibit metastases more effectively than when amiloride is used alone.

NaHCO3 (Arm & Hammer Baking Soda) was used daily, in some cases, at a dose ranging from 5 grams to 30 grams per day, or more. Frequent breaks and lower of dose was necessary due to bloating and abdominal discomfort.

Intravenous lactate and/or glucose (with or without insulin) are useful for potentiating this therapy. The glucose and insulin help increase glycolysis in cancer cells, increasing intracellular acidity. In some embodiments, an IV with high-dose glucose and Na lactate is administered. The lactate can impede the activity of the MCT and also reverse, at least partially, the gradient of high intracellular to extracellular lactate. In some embodiments, insulin is not administered due to patient discomfort.

Some patients are also treated with daily probiotics and digestive enzymes (high dose PPI along with NAHCO3 can cause difficulty digesting proteins).

### Example 1

### Patient MN

MN is a 53-year-old female diagnosed on Aug. 5, 2010, with stage 4B endometrial carcinoma, FIGO grade 2, moderately differentiated. A laparotomy revealed invasion through myometrium to uterine serosa, where tumor was present on the serosal surface and in fibrous adhesions of the serosa, with involvement of the lower uterine segment. Bilateral adnexa revealed deposits of adenocarcinoma. Omentum was found to have focal deposits of metastatic carcinoma. The patient had a debulking procedure, the surgeon being unable to resect remaining cancer invading bowel and bladder. The patient was scheduled for chemotherapy, Cisplatin, Taxol, Doxorubicin (standard of care for stage 4B), but refused. Her tumor markers on Sep. 13, 2010: CA-19.9 was 44 (normal <37) and CA 125 was 23 (normal <21).

On Sep. 10, 2010, MN began taking esomeprazole 40 mg tabs, 3 tabs 2 times per day, every other day. She also began taking methazolamide 50 mg tabs, 2 times per day. In addition, MN took 1 tsp Arm & Hammer Baking Soda 3 times per day. MN was also placed on probiotics and digestive enzymes prior to each meal. After being on this regimen for 1 week, the following protocol was instituted 2 times per week for 4 weeks. The above oral regimen was maintained throughout.

One liter of sterile water containing 175 mEQ Na lactate per liter and 175 grams dextrose per liter were infused over a 1 hour period. Blood glucose was checked (via a portable glucometer), demonstrating a blood glucose greater than 600 mg/dl. The patient was then placed in a Heckel HT-2000 Hyperthermia Unit (heats through infrared lamps). The patient's rectal temperature was raised to 104 degrees Fahrenheit over a 90 minute period, and then was maintained at that temperature for an additional 90 minutes. Another 500 ml of the dextrose/lactate solution was infused over the 1st hour of heating, followed by a liter of normal saline, which was infused over the remaining period of heating. The patient received 4 liters nasal cannula oxygen throughout the period of heating.

Side effects consisted of abdominal bloating, nausea, and extremity tingling (paresthesia). GI complaints were handled by decreasing NaHCO3 doses. Tingling was treated with breaks from the methazolamide.

Tumor markers at follow-up:

Oct. 11, 2010—CA 19-9 was 29; CA 125 was 5

Oct. 25, 2010—CA 19-9 was 17; CA 125 was 5

Nov. 18, 2010—CA 19-9 was 15; CA 125 was 5

Jan. 4, 2011—CA 19-9 was 13; CA 125 was 4

Feb. 7, 2011—CA 19-9 was 18; CA 125 was 5

Apr. 4, 2011—CT with and without contrast of chest, abdomen, and pelvis—normal, except for postoperative changes.

### Example 2

### Patient SL

Patient SL is a 52-year-old female diagnosed with stage 4 non-small cell lung carcinoma, presenting as a mediastinal mass with metastases to bilateral adrenal glands and spleen on Nov. 23, 2010. SL's presenting symptoms were posterior chest and upper back pain, cough, and shortness of breath. Her initial CEA on Dec. 2, 2010 was 952.1, just prior to beginning treatment with cisplatin, Alimta, and Aflibercept (a VEGF inhibitor), as well as radiation therapy. Repeat CT on Dec. 10, 2010 revealed a decrease in size of mediastinal mass. Her CEA on Feb. 28, 2011 was 309.9. A break was given from chemotherapy and an oral regimen was begun using the following:

Esomeprazole 40 mg tabs, 3 tabs 2 times per day every day; Methazolamide 50 mg tabs, slowly titrating up to a max of 200 mg 2 times per day every day to every other day (frequent breaks were taken due to nausea); Amiloride 10 mg 2 times per day; HCTZ 25 mg 2 times per day. NaHCO3 was initially used but eventually terminated due to bloating. Digestive enzymes and probiotics were also initiated.

The hyperthermia/infusion protocol listed for patient SL was initiated 1 week after starting the above oral regimen. After 2 infusions/hyperthermia sessions, the patient's cough completely resolved. Tumor markers began rising; CEA on Mar. 11, 2011 was 358.2. The patient, however, felt well and appeared clinically well, and her cough had resolved. At this time, esomeprazole 40 mg was increased to 4 tabs 2 times per day every day, and hyperthermia sessions were changed from two 3 hour sessions per week to one 6 hour session per week. CEA on Mar. 21, 2011 was 397.1, but the patient felt well. Each treatment was associated with exacerbation of the right posterior chest/upper back pain, which would subside (but not disappear) the following day. Tumor marker on Mar. 28, 2011 was 360.6, which revealed the first drop since beginning pH manipulation therapy.

The patient experienced severe nausea and vomiting on March 26, prompting a visit to the emergency room (ER). CBC and comprehensive metabolic panel were within normal limits, except for anemia (Hgb 9.3 following hydration), most likely reflecting residual bone marrow suppression from chemotherapy. Chest x-ray (CXR) was read by the radiologist as no apparent disease (NAD). Endoscopy revealed no pathology and the patient was dismissed the following day feeling well.

Apr. 4, 2011—Computed Tomography (CT) Scan of Chest, Abdomen, and Pelvis: Left upper lobe density is stable from Jan. 28, 2011; PET/CT would be required to assess activity.

New 15 mm cavitating density RLL; PET/CT would be required to further evaluate (this lesion corresponds with patient's pain during infusion/hyperthermia).

Splenic lesion decreased in size from 4.4×2.4 cm to 3.2×1.9 cm

Fullness to bilateral adrenal glands stable since Jan. 28, 2011

On visits in April 2011, patient says she feels “wonderful.”

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Throughout this application, various publications (patent or non-patent literature) are referenced. The disclosures of these publications in their entireties are incorporated by reference herein.

1. A pharmaceutical formulation, for treating cancer in a mammal, having active ingredients comprising at least two of:
(a) a monocarboxylate transport inhibitor;
(b) a sodium-hydrogen exchange inhibitor;
(c) a chloride-bicarbonate exchange inhibitor;
(d) a carbonic anhydrase inhibitor; or
(e) a proton pump inhibitor;
wherein those of (a) through (e) that are in the formulation are in amounts effective in combination to induce selective cytotoxicity in cancer cells relative to noncancerous cells in members of the same species as the mammal;
wherein the formulation is in a form enterally administrable to the mammal and in a dose that, when administered one to four times daily, is sufficient to produce a metabolic acidosis in members of the same species as the mammal and having normal renal function.

2. The formulation of claim 1, wherein the proton pump inhibitor comprises at least one of omeprazole, lansoprazole, dexlansoprazole, esomeprazole, pantoprazole, rabeprazole, dorafem, or a bafilomycin.

3. The formulation of claim 1, wherein the monocarboxylate transport inhibitor comprises at least one of lonidamine, cinnamate, a-cyano-4-hydroxycinnamate (4-CIN), or a pharmacologically active derivative of 4-CIN.

4. The formulation of claim 1, wherein the sodium-hydrogen exchange inhibitor comprises at least one of amiloride, EIPA, or another pharmacologically active derivative of amiloride.

5. The formulation of claim 1, wherein the carbonic anhydrase inhibitor comprises at least one of methazolamide or acetazolamide.

6. The formulation of claim 1, wherein the chloride-bicarbonate exchange inhibitor comprises at least one of trifolcin, DIDS, diphenylamine-2-carboxylate, s3075, or levetiracetam.

7. The formulation of claim 1, wherein the chloride-bicarbonate exchange inhibitor comprises at least one of DIDS or a pharmacologically active derivative thereof.

8. The formulation of claim 1, in a form that is enterally administrable to the mammal.

9. The formulation of claim 1, comprising the carbonic anhydrase inhibitor and the proton pump inhibitor.

10. The formulation of claim 1, comprising the monocarboxylate transport inhibitor and the carbonic anhydrase inhibitor.

11. The formulation of claim 1, comprising the proton pump inhibitor and the carbonic anhydrase inhibitor.

12. The formulation of claim 1, comprising the carbonic anhydrase inhibitor and the sodium-hydrogen exchange inhibitor.

13. The formulation of claim 1, comprising the carbonic anhydrase inhibitor and the chloride-bicarbonate exchange inhibitor.

14. The formulation of claim 1, comprising at least three of (a) through (e).

15. The formulation of claim 1, comprising at least four of (a) through (e).

16. The formulation of claim 1, comprising each of (a) through (e).

17. The formulation of claim 1, further comprising a polymeric micelle that encases the active ingredients and releases them into extracellular fluid at pH below 7.3.

18. The formulation of claim 1, wherein the active ingredients are at least partially surrounded by three layers;
wherein the first layer is the outermost of the layers and comprises a first material that is (a) substantially insoluble in aqueous media below a pH of about 5.0, and (b) substantially soluble in aqueous media above a pH of about 6.0;
wherein the second layer lies between the first and the third layer and comprises a second material that erodes at a predetermined rate in aqueous media between a pH of about 7.2 and about 7.6;
wherein the third layer is the innermost of the three layers and is configured (a) not to erode above a pH of about 7.4, and (b) to erode below a pH of about 7.3, thereby releasing the active ingredients to a target tissue of the animal from within the third layer.

19. A method, of treating cancer in a patient, comprising:
(i) administering to a patient having cancer a substance that, at a therapeutic dose, produces a metabolic acidosis in humans; and
(ii) administering to the patient at least one of:
(a) a monocarboxylate transport inhibitor;
(b) a sodium-hydrogen exchange inhibitor;
(c) a chloride-bicarbonate exchange inhibitor; or
(d) a proton pump inhibitor;
wherein the at least one of (a) through (d) is in an amount effective to induce selective cytotoxicity in cancer cells relative to noncancerous cells in humans.

20. The method of claim 19, wherein the substance comprises a carbonic anhydrase inhibitor.
